(19) United States
(12) Patent Application Publication
(10) Pub. No.: US 2026/0364340 A1
(43) Pub. Date: Nov. 19, 2026

(54) TARGET TISSUE ENGAGEMENT

(71) Applicant: Inspire Medical Systems, Inc., Golden Valley, MN (US)

(72) Inventors: Kevin Verzal, Lino Lakes, MN (US); Natasha Marie Kuntz, Champlin, MN (US); Kristen Marks Ragnacci, Hopkins, MN (US); Nathan Olson, Shoreview, MN (US)

(73) Assignee: Inspire Medical Systems, Inc., Golden Valley, MN (US)

(21) Appl. No.: 19/470,884

(22) PCT Filed: Mar. 28, 2024
(86) PCT No.: PCT/US2024/022047

§ 371 Date: Sep. 30, 2025

Related U.S. Application Data
(60) Provisional application No. 63/456,134, filed on Mar. 31, 2023.

(51) Int. Cl. A61N 1/05; A61N 1/36

(52) U.S. Cl. CPC A61N 1/0556; A61N 1/0558; A61N 1/3601

(57) ABSTRACT

Implantable medical apparatuses and methods configured to engage target tissue, such a nerve, via a first electrode arrangement. Such engagement may, in some examples, include improving, increasing, and/or otherwise enhancing electrical capture of the target tissue, such as a nerve, for stimulation and/or sensing. In some examples, the first electrode arrangement may be previously implanted.

### BACKGROUND

Some chronically implantable medical devices may include an electrode for sensing or stimulating purposes of a nerve or other target tissue.

### BRIEF DESCRIPTION OF THE DRAWINGS

FIG. 1A is a diagram schematically representing an example implantable medical apparatus.

FIG. 1B is a diagram including a front view schematically representing a patient's body, implantable components, and/or external elements of example methods and/or example devices.

FIG. 2A is a diagram including a front view schematically representing a patient's body, implantation path, and example implanted medical device.

FIG. 2B is a plan view schematically representing an example stimulation portion including a first electrode arrangement.

FIG. 2C is a schematic diagram of a control portion.

FIG. 3A is front view of an example of an implantable medical apparatus configured to receive a first electrode arrangement, the implantable medical apparatus having a second electrode arrangement and a third electrode arrangement (showing the third electrode arrangement in cross-section for ease of illustration).

FIG. 3B is a partial, front view of the example first electrode arrangement and third electrode arrangement of the implantable medical apparatus of FIG. 3A in a disengaged arrangement.

FIG. 3C is a front view of the example first electrode arrangement and third electrode arrangement of the implantable medical apparatus of FIG. 3A in an engaged arrangement.

FIG. 4 is front view of example first and third electrode arrangements akin to those FIGS. 3A-3C, except with seal portions on the first electrode arrangement instead of on the third electrode arrangement.

FIG. 5A is front view of an example of an implantable medical apparatus configured to receive a first electrode arrangement, the implantable medical apparatus having a second electrode arrangement and a third electrode arrangement (showing the third electrode arrangement in cross-section for ease of illustration).

FIG. 5B is a front view of an electrode arrangement that can be utilized with any of the implantable medical apparatuses of the disclosure according to one example.

FIG. 6A is front view of an example of an implantable medical apparatus configured to receive a first electrode arrangement, the implantable medical apparatus having a second electrode arrangement and a third electrode arrangement.

FIG. 6B is a partial, sectional view of the first electrode arrangement and third electrode arrangement of the implantable medical apparatus of FIG. 6A in a disengaged relationship.

FIG. 6C is a front view of the first electrode arrangement and third electrode arrangement of the implantable medical apparatus of FIG. 6A-6B in an engaged relationship.

FIG. 6D is front view of an example of an implantable medical apparatus configured to receive a first electrode arrangement, the implantable medical apparatus comprising a second electrode arrangement and a third electrode arrangement (showing the third electrode arrangement in cross-section for ease of illustration) with the third electrode arrangement and the first electrode arrangement in an example disengaged relationship.

FIG. 6E is a partial, sectional view of the second electrode arrangement and third electrode arrangement of the implantable medical apparatus of FIG. 6D in an example engaged relationship.

FIGS. 6F, 6G are each a side sectional view of the first electrode arrangement and third electrode arrangement of the implantable medical apparatus of FIG. 6D in an example disengaged arrangement and example engaged relationship, respectively.

FIG. 6H is a top plan view of an example first electrode arrangement positioned relative to an example third electrode arrangement.

FIG. 6I is a top plan view of an example third electrode arrangement.

FIG. 6J is a top plan view illustrating an example first electrode arrangement engaging a third electrode arrangement.

FIG. 6K is a top plan view of an example first electrode arrangement.

FIGS. 6L, 6M are side sectional views illustrating an example third electrode arrangement and first electrode arrangement in a disengaged relationship and engaged relationship, respectively.

FIG. 7 is a front view of an example of an implantable medical apparatus comprising a wrap engaging a nerve and electrode arrangement.

FIGS. 8-9 are sectional views taken along lines 8-8, lines 9-9 of FIG. 7 representing example wraps.

FIG. 10A is a front view of an example implantable medical apparatus akin to FIG. 7, with the wrap including electrodes to engage the nerve and the electrode arrangement.

FIGS. 10B and 10C are sectional views taken along lines 10B-10B, lines 10C-10C of FIG. 10A representing example wraps including electrodes.

FIGS. 11A-11B are front views of examples of implantable medical apparatuses comprising a plurality of wraps.

FIGS. 12-18 collectively illustrate examples of an implantable medical apparatus including a wrap that can be positioned over an electrode arrangement and maintained in position thereto with one or more clips that can be secured to a respective retainer on the electrode arrangement.

FIG. 19 is a perspective view of an example of an implantable medical apparatus including a plurality of wraps.

FIGS. 20-21 are schematic cross-sectional views of examples of wraps having a plurality of electrodes.

FIG. 22 is a schematic view of an example of an implantable medical apparatus connected to a nerve and secured in position with an anchor.

FIG. 23A is a schematic view of an example of an implantable medical apparatus.

FIG. 23B is a schematic view of an example of the implantable medical apparatus of FIG. 23A including an atraumatic tip.

FIGS. 24-25 are schematic views of an implantable medical apparatus wrapped around a nerve according to examples of the disclosure.

FIG. 26 is a schematic view of an implantable medical apparatus wrapped around a nerve according to an example of the disclosure.

FIG. 27 is a schematic illustration of two implantable medical apparatuses that can be secured to a pulse generator at two different locations.

FIG. 28 schematically represents an example of an electrode arrangement, including individually addressable contact electrodes, in stimulating relation to target tissues.

FIG. 29A is a diagram schematically representing an example method and/or example device for treating sleep disordered breathing via an implantable medical apparatus including an electrode arrangement including a connected array of stimulation elements, each of which are independently positionable into stimulating relation to at least different portions of upper airway patency-related tissues, including nerve portion(s).

FIG. 29B is a sectional view schematically representing an example lead segment including multiple signal electrical conductors extending within and through at least a portion of the lead segment.

FIG. 29C is a top plan view schematically representing an example portion of a lead body for strain relief and/or other purposes.

FIGS. 29D-29E are diagrams including top and side views schematically representing example stimulation elements including a linear electrode array on a paddle-style carrier.

FIGS. 29F-29G are diagrams including top and side views schematically representing example stimulation elements including a linear electrode array on a cylindrically-shaped carrier.

FIG. 29H is a sectional view as taken along lines 26B-26B of FIG. 26B of an example cuff electrode.

FIG. 29I is a side view schematically representing an example cuff electrode.

FIG. 30A is a front view schematically representing an example electrode arrangement including externally located electrode(s) and FIGS. 30B, 30C are sectional views as taken along lines 30B-30B, 30C-30C of FIG. 30A.

FIGS. 30D, 30E each are a front view illustrating example electrode arrangements including an extension supporting an external electrode(s).

FIGS. 31A-33 are flow diagrams and block diagrams of example methods of the disclosure.

FIG. 34A is a diagram including a front view schematically representing a patient's body, implantable components, and/or external elements of example methods and/or example devices.

FIG. 34B is a diagram schematically representing example patient anatomy and example locations for stimulating an IHM-innervating nerve and/or hypoglossal nerve.

FIGS. 35A-35B are block diagrams schematically representing an example control portions.

FIG. 35C is a block diagram schematically representing an example user interface.

FIG. 36 is a block diagram schematically representing example communication arrangements between a medical device and other devices, which may or may not comprise medical devices.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to devices for diagnosis, therapy, and/or other care of medical conditions throughout the body. At least some examples may comprise implantable devices and/or methods comprising use of implantable devices. In some examples, the implantable devices may comprise elements for delivering stimulation (e.g. a stimulation element). In some examples, such elements also may be used for sensing, whether instead of stimulation or in addition to stimulation.

In some examples, the stimulation element may include a pulse generator, which may be implemented as a microstimulator in some examples. In some examples, the pulse generator may comprise an implantable component(s), an external component(s), or a combination thereof.

At least some of the example devices and/or example methods may relate to sleep disordered breathing (SDB) care, which may comprise monitoring, diagnosis, and/or stimulation therapy.

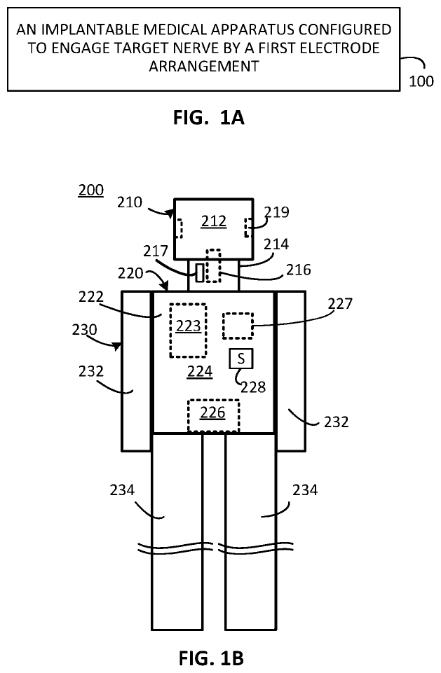

As represented at 100 in FIG. 1A, at least some aspects of the present disclosure relate to implantable medical apparatuses and methods to electrically engage target tissue, which may comprise nerve, muscle, or other tissue as also described elsewhere herein. Engaging target tissue may, in some examples, include improving, increasing, and/or otherwise enhancing electrical capture of the target tissue such as via an electrode arrangement in which an electrically non-conductive carrier supports and provides at least one electrode to be in electrical relation (e.g. stimulating relation, sensing relation, etc.) with the target tissue. In some instances, the electrode arrangement may sometimes be referred to as an electrode-carrier arrangement.

In some examples, the implantable medical apparatus may be implanted in a supplemental procedure, post-implant of a previously implanted electrode arrangement and, perhaps, other auxiliary components/devices. In various examples, the implantable medical apparatus may utilized to address poor or inadequate performance of the previously implanted electrode arrangement, which could be due to post-implantation drifting of the previously implanted electrode arrangement and/or other reasons. For example, the previously implanted electrode arrangement may not sufficiently or reliably enough electrically capture the target nerve for stimulation and/or for sensing purposes. In some examples, a lack of capture may be determined via testing during an implantation procedure or determined later.

Among other effects, electrode arrangements which do not enable a signal to sufficiently capture a nerve(s) may result in the patient being relatively non-responsive to delivered stimulation therapy. Such deficiencies may lead to uncertainty about whether the particular patient is a poor candidate for the designated stimulation therapy, whether the implantation for a specific patient was less than optimal, whether the particular electrode arrangement of the implanted stimulation element may be less than robust in terms of capturing target tissue with a stimulation signal, and the like. Similarly, to the extent that a stimulation element including an electrode arrangement (e.g. including a plurality of electrodes) may also be used for sensing (or exclusively used for sensing), poor electrical communication between a target tissue (e.g. nerve) and electrodes of the implanted electrode arrangement may result in poorly sensed physiologic phenomenon of interest.

Accordingly, in some examples of the present disclosure, an implantable medical apparatus including a stimulation element may be implanted in association with a previously implanted stimulation element to enhance electrical engagement of the target tissue.

However, some examples of the present disclosure may increase electrical engagement with target tissue without additionally (e.g. later) implanting a second electrode arrangement. For instance, a particular type (or size, shape, etc.) of previously implanted electrode arrangement (e.g. axial-style electrode array, paddle array) may have been believed to be a good match for a particular location, type of nerve, etc., but too much space exists between the previously implanted electrode arrangement and the target tissue. This excess space may be due to drift over time (after implantation) and/or other factors relating to the style of electrode arrangement, scar tissue encapsulation, lead movement, associated anchors, etc. Accordingly, one example implantable medical apparatus may comprise implantable components to mechanically urge an electrode arrangement(s) of a previously implanted stimulation element into close proximity to a target tissue (e.g. target nerve), which in turn may increase electrical engagement of the electrode arrangement relative to the target nerve(s). In some of these examples, the term “close proximity” may comprise the additionally implanted electrode arrangement becoming robustly secured against a surface of the target tissue and/or becoming spaced part from the target tissue at a distance small enough that a desired level of electrical capture (e.g. for suprathreshold stimulation) may be achieved.

In some examples, implementing electrical engagement relative to target tissue may comprise achieving electrical capture relative to the target tissue where there was no electrical capture previously, such as (but not limited to) engaging a target tissue which did not previously have a stimulation element (e.g. including an electrode arrangement) placed into stimulating relation to the particular target tissue. In some of these examples, the location at which an additional electrode arrangement is introduced (e.g. implanted) may comprise nerve branches related to, extending from, etc. the nerve branch on which the previously implanted electrode arrangement is located. In some of these examples, the additionally placed second electrode arrangement may be added on the same side of the body as a previously placed first electrode arrangement (e.g. unilateral deployment). However, in some of these examples, the additionally placed second electrode arrangement may be added on a different side of the body relative to the previously implanted electrode arrangement, thereby achieving bilateral deployment.

In some examples, an initial implantation for a patient may include implantation of an arrangement configured to be secured relative to, and to place electrode arrangement(s) in stimulating relation (and/or sensing relation) to, multiple nerve branches.

These examples, and additional examples, are further described in association with at least FIGS. 1A-36.

Referring now to FIG. 1B regarding features in addition to, or instead of, the features associated with FIG. 1A, FIG. 1B is block diagram schematically representing a patient's body 200, including example target portions 210-234 at which at least some example stimulation elements (and/or sensing elements) may be employed to implement at least some examples of the present disclosure.

As shown in FIG. 1B, patient's body 200 comprises a head-and-neck portion 210, including head 212 and neck 214. Head-and-neck portion 212 comprises cranial tissue, nerves, etc., and upper airway 216 (e.g., nerves, muscles, tissues), etc. As further shown in FIG. 1B, the patient's body 200 comprises a torso 220, which comprises various organs, muscles, nerves, other tissues, such as but not limited to those in pectoral region 222 (e.g., lungs 223, cardiac 227), abdomen 224, and/or pelvic region 226 (e.g., urinary/bladder, anal, reproductive, etc.). As further shown in FIG. 1B, the patient's body 200 comprises limbs 230, such as arms 232 and legs 234.

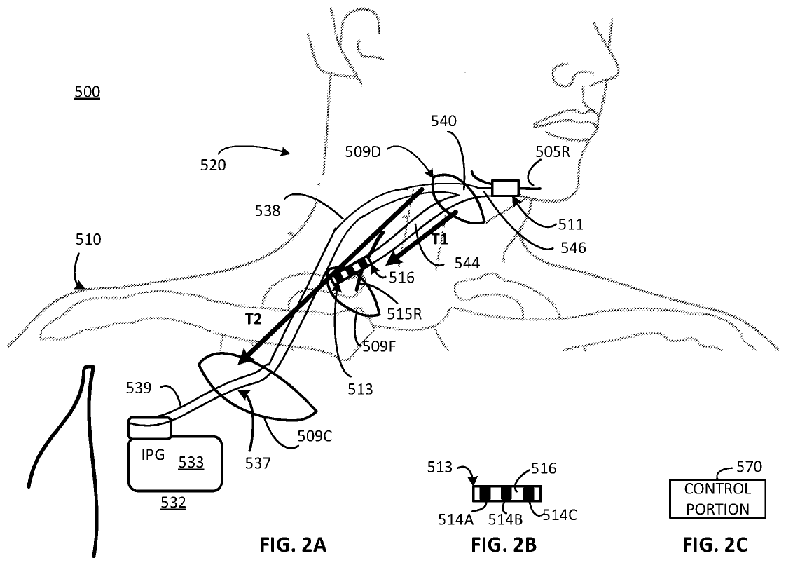

It will be understood that various sensing elements and/or stimulation elements as described throughout the various examples of the present disclosure may be deployed within the various regions of the patient's body 200 in order to sense and/or otherwise diagnose, monitor, treat various physiologic conditions such as, but not limited to those examples described below in association with FIGS. 2A-36. In some such examples, a stimulation element 217 may be located in or near the upper airway 216 for treating sleep disordered breathing and/or a sensing element 228 may be located anywhere within the neck 214 (and/or head-and-neck region) and/or torso 220 (or other body regions) to sense physiologic information for providing SDB care including, but not limited to, sensing respiration, sleep, and/or related parameters.

In some examples, at least a portion of the stimulation element 217 may comprise part of an implantable component/device, such as an implantable pulse generator (IPG) whether full sized (e.g. for pectoral implantation) or sized/shaped for implantation in the head-and-neck portion 212 such as in neck portion 214. In some such examples, the smaller sized/shaped IPG may sometimes be referred to as a microstimulator. The implantable components (e.g. IPG, other) may comprise a stimulation/control circuit, a power source (e.g. non-rechargeable, rechargeable), communication elements, and/or other components. In some examples, the stimulation element 217 also may comprise a stimulation electrode and/or stimulation lead connected to the implantable pulse generator.

In some examples, the IPG is sized and/or shaped for implantation in the neck portion 214 (versus a larger IPG suitable for pectoral implantation but not neck portion 214) and comprises a housing sealingly containing the power source, stimulation circuitry, control circuitry, sensing circuitry, internal sensing elements (e.g. accelerometer, other), and/or communication elements. In some such examples, this smaller sized/shaped IPG may comprise, and/or may sometimes be referred to as, a microstimulator. In some examples, this smaller sized/shaped IPG (e.g. microstimulator) also may comprise elements mounted on its external surface for sensing and/or stimulation, and/or may comprise a lead extending from (and/or part of) a housing of the IPG with such leads comprising sensing elements (e.g. electrodes, other) and/or stimulation electrodes.

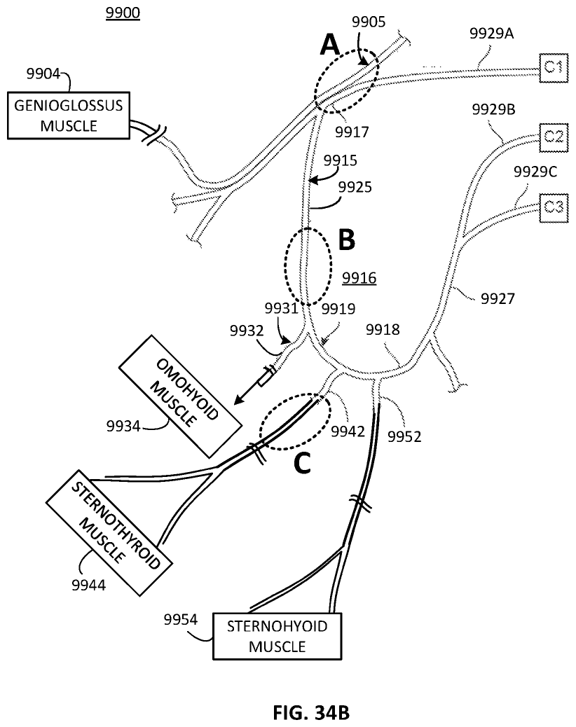

In some examples, at least a portion of the stimulation element 217 may comprise part of an external component/device such as, but not limited to, the external component comprising a pulse generator or at least some pulse generation components (e.g. stimulation/control circuitry), power source (e.g. rechargeable, non-rechargeable), and/other components. In some examples, a portion of the stimulation element 217 may be implantable and a portion of the stimulation element 217 may be external to the patient. Similarly, in some examples, a portion of sensing element 228 and/or additional sensing elements may be located external to the patient. At least some aspects of such external components are further described later in association with at least FIG. 34.

Further details regarding a location, structure, operation, and/or use of the sensing element 228 and/or stimulation element 217 are described below in association with at least FIGS. 2A-36. Among some examples, the example arrangement in FIG. 2A provides one example of a chronically implanted stimulation element(s), including an implantable pulse generator, as further described below.

It will be further understood that the examples of the present disclosure may be implemented in many different parts of the body 200 and/or to implement care (e.g. sensing, monitoring, diagnosis, stimulation, etc.) for a wide variety of physiologic conditions, and thus the examples are not limited to sleep disordered breathing (SDB) care. Among other locations and/or physiologic conditions, at least some examples of the present disclosure may be implemented in a pelvic region to treat pelvic disorders such as, but not limited to, various forms of incontinence (whether fecal or urinary) or may be implemented for pain management (e.g., lower back).

To facilitate appreciation of at least some of the ways in which examples of the present disclosure may be implemented, FIG. 2A schematically illustrates at least some aspects of one example implantation procedure and arrangement by which an implantable stimulation arrangement may be placed within a patient, and relative to which at least some examples of the present disclosure may be implemented to enhance electrical engagement of electrode arrangements relative to a target tissue (e.g. target nerve).

FIG. 2A is a diagram including a front view schematically representing an example arrangement 500 relative to a patient's body 510, including an example device for, and/or example method of, implantation of a lead 537 including a stimulation portion 511 in stimulating relation to a hypoglossal nerve 505R and a stimulation portion 513 in stimulating relation to an infrahyoid muscle (IHM)-innervating nerve 515R (later described more fully in association with at least FIG. 34B) In some examples, the example arrangement 500 may comprise at least some of substantially the same features and attributes as, comprise an example implementation of, and/or be usable with the example arrangements described in association with at least some of FIGS. 1A-1B. In some examples, each stimulation portion may sometimes be referred to as being a stimulation element or as part of a stimulation element.

As shown in FIG. 2A, the lead 537 may comprise a proximal portion 539, a body portion 538, and first and second distal portions 546, 544, which extend from body portion 538 via a junction 540. In some instances, the lead 537 may sometimes be referred to as comprising a bifurcated lead at least to the extent that the junction 540 extending from lead body portion 538 is shaped to result in bifurcation of the respective first and second distal portions 546, 544 from the lead body portion 538. Alternatively, the bifurcation point may be located in the pectoral region, or two separate leads could extend from the generator to the target implant sites.

As shown in FIG. 2A, the various components of the example arrangement 500 are implantable via at least implant-access incision 509C and via implant-access incision 509D in neck 520. In some examples, upon forming implant-access incision 509C, IPG 533 may be implanted subcutaneously, such as in a subcutaneous pocket within pectoral region 532. In some examples, an implant-access incision 509F is also formed to enable implantation of components of example arrangement 500 such as, but not limited to, stimulation portion 513. In some examples, the stimulation portion 513 may comprise an axial-style electrode arrangement (e.g. series of spaced apart, split ring or ring electrodes 514A, 514B, 514C supported on/by a nonconductive carrier body 516, which may include one or more individual components such as nonconductive rings positioned between the electrodes 514A-514C).

Upon forming implant-access incision 509D, a distal portion of lead 537 can be implanted subcutaneously, which may include several aspects.

In one aspect, the first distal portion 546 of lead 537 may be implanted subcutaneously, via implant-access incision 509D, and advanced until stimulation portion 511 is in stimulating relation to nerve 505R. In particular, first distal portion 546 (including stimulation portion 511) may have a length which is sufficiently short such that first distal portion 546 (including stimulation portion 511) may be implanted with little or no tunneling from implant-access incision 509D. Stated differently, the location of implant-access incision 509D may be selected in sufficiently close proximity to the target stimulation location along nerve 505R such that little or no tunneling (from implant-access incision 509D to the target stimulation site) is performed to implant first distal portion 546 (including stimulation portion 511) in stimulating relation to (a target stimulation location) of nerve 505R.

In another aspect, prior to implanting a second distal portion 544 of lead 537, tunneling (as represented by arrow T1) may be performed from implant-access incision 509D toward nerve 515R. Thereafter, the second distal portion 544 (including stimulation portion 513) may be advanced via the tunnel (e.g. T1) to place stimulation portion 513 in stimulating relation to nerve 515R.

In one aspect, a proximal portion 539 of the body portion 538 of lead 537 is to be implanted to extend toward and into connection with IPG 533. However, in some examples, tunneling is first performed between the implant-access incision 509D and implant-access incision 509C to establish a tunnel (i.e. pathway), as represented by arrow T2). It will be understood that the tunneling may be performed starting at either implant-access incision 509C, 509D. With the tunnel in place, the proximal portion 539 of lead 537 is inserted and advanced through implant-access incision 509D toward IPG 533 until the body portion 538 extends from the implant-access incision 509D to implant-access incision 509C, at which the proximal portion 539 of lead 537 may be further maneuvered to be electrically and mechanically connected to the IPG 533.

In some examples, a separate lead may be provided for each respective stimulation portion 511 and stimulation portion 513, with each separate lead extending (e.g. directly) from the IPG 533. In some such examples, a single implant-access incision (e.g. 509C) may be located at/near the target implant location of the IPG 533 and provide a starting point from which tunneling (e.g. separate tunnels or a single tunnel) may be performed to the target implant location for each of the respective stimulation portions 511 (e.g. at 505R), 513 (e.g. at 515R). Alternatively, in some such examples, a single implant-access incision (e.g. 509D) may be located at/near the target implant location of the stimulation portion 511 and provide a starting point from which tunneling (e.g. separate tunnels or a single tunnel) may be performed to the target implant location for each of the respective stimulation portion 513 (e.g. at 515R) and IPG 533 (e.g. in pectoral region). In some such examples, a single implant-access incision (e.g. 509F) may be located at/near the target implant location of the stimulation portion 513 and provide a starting point from which tunneling (e.g. separate tunnels) may be performed in opposite directions to the target implant location for each of the respective stimulation portion 513 (e.g. at 515R) and IPG 533 (e.g. in pectoral region).

It will be further understood that, in some examples, the particular sequence in which the various aspects of implantation (e.g. first distal portion 546, second distal portion 544, body portion 538, IPG 533) are performed may vary depending on the circumstances, preferences, etc.

It will be further understood that at least some of the various lead body portions shown in FIG. 2A may further comprise variable length features (e.g. sigmoid shape, sinusoidal shape, other) which may provide strain relief, among other properties. Similarly, it will be further understood that at least some of the various lead body portions may be anchored relative to non-nerve tissues.

As addressed elsewhere, while FIG. 2A depicts the stimulation portion 511 (for right hypoglossal nerve 505R) and the stimulation portion 513 (for right infrahyoid-muscle (IHM) innervating nerve 515R) on a same side of the body, it will be understood that example arrangement 500 may be implemented such that a respective one of the stimulation portions (511, 513) may be implanted on a first side (e.g. right or left side) of the patient's body and the other respective one of the stimulation portions (511, 513 may be implanted on an opposite, second side (e.g. right or left side) of the patient's body. In some such examples, at least one of the tunnels T1 and/or T2 (or other tunnels) and a lead portion (e.g. 544 and/or 538) may extend across a sagittal midline of the patient's body, such along the neck below a patient's chin.

Moreover, at least some example arrangements may be implemented so that both the left and right hypoglossal nerves and both the left and right IHM-innervating nerves have their own stimulation portion (e.g. 4 different locations) such that stimulation may be applied in any desired combination from among a right side hypoglossal nerve, a left side hypoglossal nerve, a right side IHM-innervating nerve, and a left side IHM-innervating nerve. However, of course, the fact that each of the 4 different locations may have an implanted stimulation portion does not necessitate that stimulation occurs at any one of the given locations at least because at least some example stimulation protocols, methods, etc. may comprise selective stimulation among multiple implanted stimulation portions in order to achieve various goals in treating sleep disordered breathing.

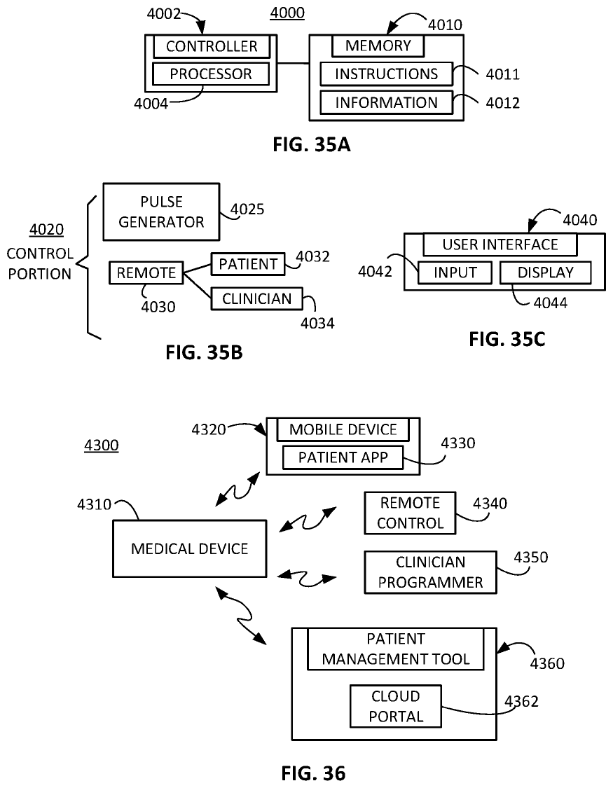

In some examples, the IPG 533 may comprise a control portion 570 as shown in FIG. 2B, with control portion 570 comprising at least some of substantially the same features and attributes as the control portion 4000 in FIGS. 35A-36. The control portion 570 may be used to implement at least some of the various example devices and/or example methods of the present disclosure as described herein, such as to implement stimulation, sensing, and/or other functions.

It will be understood that the particular example arrangement of lead portions 546, 544 of lead 537, stimulation portions 511, 513, nerves 505R, 515R, etc. shown in FIG. 2A is just one example of a wide range of different types of lead portions, different types of stimulation elements, different nerve types, different locations, etc. by which implantation may occur. Therefore, further examples described throughout the present disclosure (e.g. via FIGS. 1A-1B, 2B-36) are not strictly limited to the example arrangement of FIG. 2A. For instance, in some examples, a previously implanted medical device may comprise lead portion 544 without lead portion 546, or vice versa, such that the sole implanted lead portion may be in operative relation (e.g. stimulating relation and/or sensing relation) with just one target tissue in some examples. Moreover, in some further examples, the IPG may be sized and/or shaped to be implanted in the neck region 520 (e.g. via an incision located in the neck region 520), with lead body 538 having a significantly shorter length, and with just one or both of lead portions 544, 546.

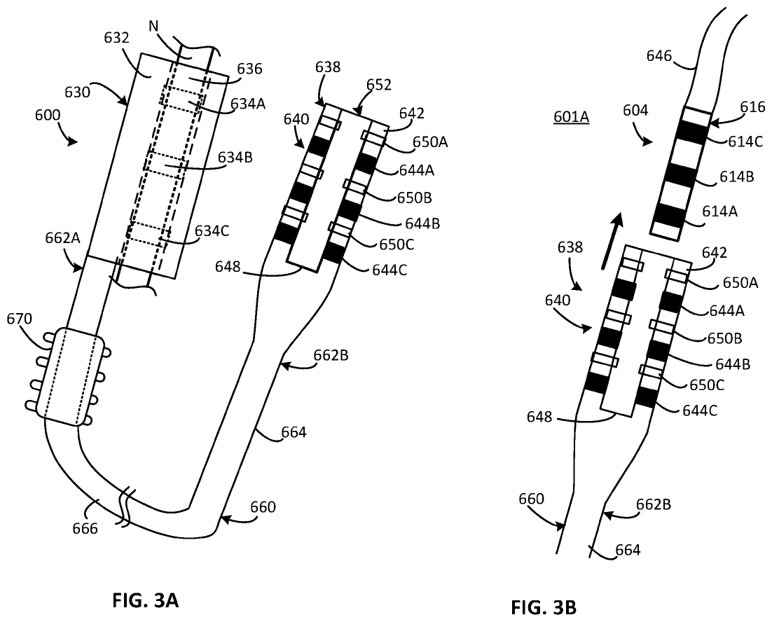

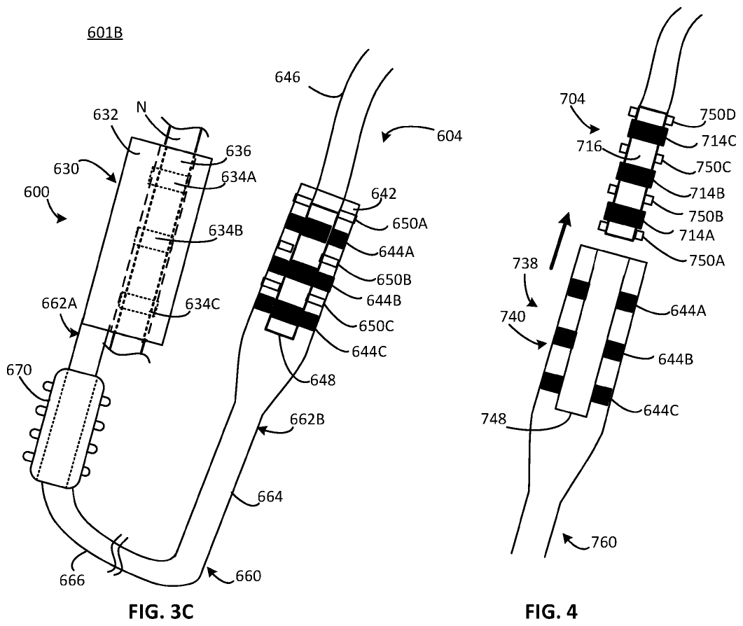

With this in mind, an example arrangement in FIGS. 3A-3C is provided for use with a previously implanted electrode arrangement (e.g. first electrode arrangement) such as (but not limited to) a stimulation element like stimulation portion 513 in FIG. 2A.

FIGS. 3A-3C illustrate one example implantable medical apparatus 600 formed of biocompatible materials appropriate for implantation into the human body. In some examples, the apparatus 600 comprises an example implementation of, and/or at least some of substantially the same features as, the example arrangements of FIGS. 1A-1B, 2A, 2B, 2C. In some examples, the implantable medical apparatus 600 is configured to increase a degree of stimulating relation or sensing relation between a first electrode arrangement 604 and a target nerve T. In various examples, at least some components of the implantable medical apparatus 600 are configured to transition from a disengaged arrangement 601A (FIG. 3B) to engage or otherwise be removably/releasably connected with the first electrode arrangement 604 in an engaged arrangement 601B (FIG. 3C).

In one example, as shown in FIG. 3B, a stimulation element includes the first electrode arrangement 604 which includes a lead 646 connected to a plurality of electrodes 614A, 614B, 614C maintained by a non-conductive carrier body 616 in an electrically isolated manner. In some examples, the electrodes 614A, 614B, 614C may sometimes be referred to as contact electrodes or stimulation electrodes and the electrodes also may sometimes be referred to as sensing electrodes, depending on the context in which the electrodes may be deployed.

Each of the plurality of electrodes 614A-614C are formed of an electrically conductive material appropriate for delivering stimulation and/or sensing energy within the human body. The plurality of spaced apart electrodes 614A-614C are arranged along the carrier body 616 so as to provide an exposed surface (from which stimulation/sensing energy is emitted) at or relative to the outer surface of the carrier body 616. The carrier body 616 may be flexible to increase patient comfort and allow for adaptation to varying patient anatomy according to various examples or may otherwise be inflexible. The U-configuration illustrated is just one example of a flexible or inflexible trajectory.

As further shown in FIGS. 3A-3C, in one example, the implantable medical apparatus 600 includes a second electrode arrangement 630 comprising a carrier body/non-conductive element 632 which supports a plurality of electrodes 634A, 634B, 634C (e.g., stimulation electrodes). The second electrode arrangement 630 can include at least some of substantially the same features or properties of the first electrode arrangement 604. In some examples, the non-conductive carrier 632 is configured to at least partially implement the engagement of the target nerve. For example, the non-conductive element (e.g. carrier) 632 may comprise portions configured to wrap at least partially around or otherwise contact the target nerve such the portions (e.g. flanges, flaps, fingers, etc.) of the carrier 632 at least partially define a lumen 636 through which the target nerve N extends, and the portions of the carrier 632 are sized, shaped, and biased to at least partially encircle the target nerve N. In some such examples, second electrode arrangement 630, including the non-conductive carrier 632 and plurality of electrodes 634A, 634B, 634C, may comprise a cuff electrode.

Via such example arrangements, one or more electrodes 634A, 634B, 634C of the second electrode arrangement 630 are urged into stimulating relation with the target nerve, which in some examples may comprise direct contact of the electrodes 634A, 634B, 634C with a surface of the target nerve. In some such examples, stimulating relation may comprise being in sufficiently close proximity to the surface of the target nerve in order to achieve suprathreshold stimulation of the target nerve (or select fascicles/fibers thereof), which in turn may ensure desired contraction of target muscles innervated by the target nerve, such as in the example of delivering a stimulation signal. In some of these examples, stimulating relation may comprise being in sufficiently close proximity to achieve tonal stimulation of the target nerve (or select fascicles/fibers thereof) to ensure a desired tone response of target muscles innervated by the target nerve.

In one example, the carrier body 632 is interconnected to a dock 638 that is configured to receive the first electrode arrangement 604. The dock 638 includes a third electrode arrangement 640 including a nonconductive carrier body 642 supporting a plurality of spaced-apart electrodes 644A, 644B, 644C according to an example of the disclosure. In one example, the second electrode arrangement 630 is at one terminal end 662A of a lead body extension 660 and the dock 638 is at an opposing terminal end 662B of the lead body extension 660 (i.e. the lead body extension separates and interconnects the first electrode arrangement and the dock). According to one example, the lead body extension 660 provides or carries within a non-conductive sleeve 664 a plurality of wires/conductors electrically connected to the electrodes 634A-634C, 644A-644C of both the second electrode arrangement 640 and a third electrode arrangement 640 of the dock 638. The lead body extension 660 is of sufficiently flexible construction and length for placement within a patient's body and for coupling to a pulse generator (see also FIGS. 1B-2B and related disclosure) via the first electrode arrangement 604 to deliver electrical energy to the electrodes 634A-634C, 644A-644C for stimulation and/or sensing. To receive the first electrode arrangement 604, the dock 638 can, for example, include a recess/channel 648 having only one open end, which is sized to removably/releasably receive the end of, and permit passage of a length of, the first electrode arrangement 604. The recess 648 can be cylindrical in shape but other shapes are envisioned. In one example, the third electrode arrangement 640 is configured to be in electrical communication with the first electrode arrangement 604 when the first electrode arrangement 604 is engaged within the dock 638 (FIG. 3C). For example, the first electrode arrangement 604 may be positioned within the recess 648 to achieve engagement. In some examples, each electrode 614A-614C of the first electrode arrangement 604 becomes positioned in direct correspondence and exclusive electrical connection with a corresponding electrode 644A-644C of the third electrode arrangement 640 in order to provide individual addressability of the electrodes 644A-644C of the third electrode arrangement 640 via the individual addressability of the electrodes 614A-614C of the first electrode arrangement 604. As shown in FIG. 3C, in some such examples this arrangement may comprise the contact electrodes 614A-614C of the first electrode arrangement 604 being in contact against the contact electrodes 644A-644C of the third electrode arrangement 640.

As the implantable medical apparatus 600 is implanted within a human body according to various examples of the disclosure, the dock 638 and/or first electrode arrangement 604 can include one or more seals configured to seal the connection between the dock 638 and the first electrode arrangement 604. In one example, the dock 638 can include a plurality of seals 650A, 650B, 650C, such as one or more seals positioned between two adjacent electrodes of the plurality of electrodes 614A, 614B, 614C of the third electrode arrangement 604. Via this arrangement, a given pair of adjacent electrodes (e.g., 644A, 644B) of the first and third electrode arrangements 604, 640 become electrically isolated (e.g. independent of) other pairs of electrodes of the respective first and third electrode arrangements 604, 640. In one example, one or more of the seals 650A-650C protrude into the recess 648 of the dock 638, which may ensure sealing contact against the surface of the first electrode arrangement 604, such as against the non-conductive portions of the carrier body 616 between the adjacent electrodes 614A-614C of the first electrode arrangement 604. In one example, the plurality of seals 650A-650C are provided along a length of the recess 548 of the dock 638 and at least one seal (e.g. 650A) may be provided at the opening 652 of the recess 648 to prevent fluid ingress into the recess 648 when the first electrode arrangement 604 is engaged with the dock 638.

Via the dock 638 (and its third electrode arrangement 640), the first electrode arrangement 604 becomes electrically connected to the second electrode arrangement 630 to maintain individual addressability of the electrodes 634A-634C of the second electrode arrangement 630 via control of the individual addressability of the electrodes 614A-614C of the first electrode arrangement 604 via the pulse generator 533 (and control portion 570; see also, FIGS. 2A-2B and related 23 disclosure).

In some examples, the lead body extension 660 includes a flexible, bendable/bend portion/section 666 including a pre-formed curved shape. The bendable portion 666 can, for example, be made of a material that provides flexibility or can be made of a resilient, shape-memory material such that the bend portion 666 is biased to a particular arrangement but is otherwise flexible. In some examples, the at least a portion of the lead body extension 660 may be resilient or biased to return to its original shape (which could be straight or a pre-formed curve). In one example, an outer minor dimension (e.g. width, diameter, greatest cross-sectional dimension) of the carrier body 632 can approximate that of the lead body extension 660, such that the carrier body is akin to a ribbon, cylindrical tube or a continuation of the lead body. The bendable portion 666 need not be a continuous curve but could be sinuous, U-shaped or rectangular having rounded rectangle corners according to some examples of the disclosure. In some examples, the bend portion 666 exhibits multiple translational and/or independent rotational degrees of freedom (e.g., three translational and/or three rotational degrees of freedom (including roll, yaw, pitch). In some examples, the bend portion 666 exhibits six degrees of freedom including three orthogonal rotational axes and three orthogonal translational axes. In some examples, the bend portion 666 may comprise segments that can articulate with respect to each other, which could releasably retain whatever curved shape that the clinician forms to implant the implantable medical apparatus. In one example, such bend portion 666 could be made of various materials and covered by an elastomeric jacket (e.g., a biocompatible material such as a silicone jacket or the like). Regardless of what the lead body extension 660 is formed of and whether the lead body extension 660 include a pre-formed bend portion 666 or is more linear, the biasing force to return to the original shape is low enough such that the ends 662A, 662B can be sutured or otherwise anchored relative to non-nerve tissue to retain the desired shape. For example, the desired shape may place the second electrode arrangement 630 in stimulation relation to the target nerve location and the dock 638 engaged with and in electrical communication with the first electrode arrangement 604 according to various examples of the disclosure.

In various examples, the carrier body 664/lead body extension 660 include one or more anchors 670 that may be configured to secure the implantable medical apparatus to patient tissue. In the example of FIG. 3A, one anchor 670 is provided on the lead body extension 660. In one example, the anchor 670 is on one side of the bend portion 666 (i.e. closer to the second electrode arrangement 630 than the dock 638 or closer to the dock than the second electrode arrangement). The anchor 670 can take any of a number of configurations suitable for maintaining a position of the lead body extension 660, and particularly the second electrode arrangement 630 (in some examples), with respect to the tissue to which the anchor 670 is secured.

In one example, the first electrode arrangement 604 can be an axial electrode arrangement having a plurality of ring or split ring electrodes (e.g., electrodes 614A-614C) in which the plurality of electrodes are arranged in series at distal to an end of the lead body. Though not visible in the views, individual, electrically isolated conductive wire(s) can extend from each of the plurality of electrodes 614A-614C within a thickness of the carrier body 616/646 to connect with the pulse generator (see also, FIG. 1B).

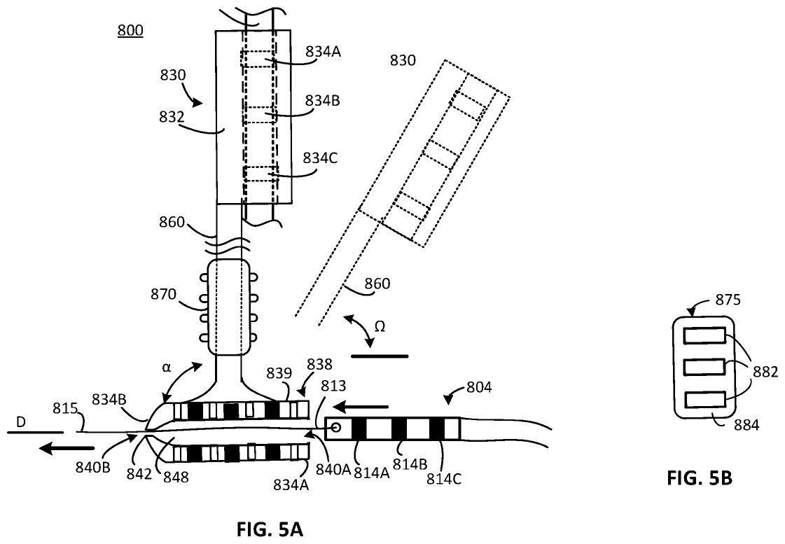

The first electrode arrangement 604 can take many configurations other than that shown in FIGS. 3B-3C. In some examples, the first electrode arrangement may include a paddle configuration that has electrodes (e.g., stimulation electrodes) arranged in a planar fashion and/or formed in multiple rows (see also, FIG. 5B). In some examples, the first electrode arrangement 604 may comprise a cuff structure, ring structure or may comprise a cylindrically-shaped element. Therefore, various examples of the disclosure include the dock 638 and/or third electrode arrangement 640 having any shape and/or size corresponding to and configured to electrically engage the first electrode arrangement 640 in a manner to maintain individual addressability of the respective electrodes 644A-644C of the third electrode arrangement 640 via individually addressing (e.g. via a control portion 570) the electrodes 614A-614C of the first electrode arrangement 604. This general arrangement, in turn, enables the electrodes 634A-634C of the second electrode arrangement 630 to be controlled via the operation and control of the electrodes of the first electrode arrangement 614A-614C. In any case, the dock 638 and third electrode arrangement 640 have corresponding structures such that the first electrode arrangement 604 can be received relative to (e.g. within in some examples) and maintained relative to (e.g. within in some examples) the dock in various examples of the disclosure.

The implantable medical apparatus 600 of FIGS. 3A-3C can be used in many ways. In one example, the implantable medical apparatus 600 may be used to engage a target nerve for stimulation and/or sensing via a first electrode arrangement. Engaging target tissue may, in some examples, include improving, increasing, and/or otherwise enhancing electrical capture of the target tissue, such as for delivering stimulation). In some examples, engaging target tissue may comprise improving, increasing, and/or otherwise enhancing electrical communication to/from the target tissue, such as for sensing parameters of/at the target tissue.

In various examples, the first electrode arrangement 604 was chronically implanted and is not producing the desired stimulation or sensing signal. In some example methods, an incision is formed in the patient (see also FIG. 2A and related disclosure) and the implantable medical apparatus 600 is positioned adjacent to the target nerve. The second electrode arrangement 630 is operatively secured to the target nerve and the first electrode arrangement 604 is engaged within the dock 638 and the third electrode arrangement 640 according to examples of the disclosure. In essence, the implantable medical apparatus 600 forms a continuation of the first electrode arrangement 604 having an electrical connection with the target nerve to engage and achieve stimulation or sensing relation (e.g. contact) with the target nerve. In some methods, the lead body extension 660 (including bendable portion 666) can be manipulated and reshaped to achieve the desired positioning of the second and/or third electrode arrangements 630, 640 with respect to the target nerve, other patient anatomy, and first electrode arrangement 604, etc. At least some examples of the disclosure can include securing the implantable medical apparatus 600 in position with one or more anchors 670 at any time during the procedure, for example, such as when the clinician feels confident that the implantable medical apparatus 600 is in the desired position and, further, when securing the implantable medical apparatus 600 in position would enhance the ease of any remaining procedure steps.

Referring in addition to FIG. 4, which illustrates an alternate implantable medical apparatus 700 and connection between a first electrode arrangement 704 and electrode arrangement 740 of a dock 738. In this example, a plurality of seals 744A, 744B, 744C, 744D are disposed on a nonconductive carrier body 716 of the first electrode arrangement 704 instead of within a recess 748 of the dock 738 as in the example of FIG. 3B. In one example, one or more seals 750A, 750B, 750C, 750D are provided between adjacent electrodes 714A, 714B, 714C, similar to the seals 650A, 650B, 650C of the example of FIGS. 3A-3C. An additional seal 750A can be provided distal to the distalmost electrode 714A and/or an additional seal 750D can be provided proximal to the proximal-most electrode 714C, distal to lead body 746, according to examples of the disclosure. Example methods of using the implantable medical apparatus of FIG. 4 can include any of the steps of the method of using the example of FIGS. 3A-3C. It is to be understood that at least some of, or all, other aspects of the implantable medical apparatus 700 can be similar or identical to that of FIGS. 3A-3C and that other features of other embodiments disclosed herein may also be incorporated into the embodiment of FIGS. 3A-3C.

Referring in addition to FIG. 5A, it will be appreciated that in some examples, it will be challenging to insert a first electrode arrangement into a dock due to the limited space available at a surgical site, adjacent bodily tissues and awkward angles in which the clinician may have to navigate. To transition the arrangement from a disengaged state of FIG. 5A in which electrodes of the first electrode arrangement are not in electrical communication with electrodes of the dock to an engaged state in which the respective electrodes are in electrical communication, the tether can be pulled to guide the first electrode arrangement into position within the dock (see also, FIG. 3C and related disclosure).

In the example of FIG. 5A, the arrangement includes an implantable medical apparatus 800 and a first electrode arrangement 804, which may be considered part of the implantable medical apparatus 800 or a separate component. It is to be understood that at least some, or all, other aspects of the implantable medical apparatus 800 and first electrode arrangement 804 can be similar or identical to that of FIGS. 3A-4 and that other features of other embodiments disclosed herein may also be incorporated into the embodiments of FIGS. 3A-4. In this example, the implantable medical apparatus 800 is configured to receive the first electrode arrangement 804. The first electrode arrangement 804 may include a carrier body/portion 816 supporting a plurality of electrodes 814A, 814B, 814C (e.g., stimulation electrodes), with the first electrode arrangement 804 being identical to electrode arrangement 604 in some examples. However, in some examples, the first electrode arrangement 804 may additionally include an elongate member 813 (e.g. tether) secured at its end 815, opposite lead body 846, adjacent the plurality of electrodes 814A, 814B, 814C (e.g., stimulation electrodes). The elongate member 813 can be a suture, wire, filament or the like, for example and can be utilized to pull the first electrode arrangement 804 into position with respect to electrode arrangement 840 of dock 838. In one example, the first electrode arrangement 804 is configured to be releasably engageable with the dock 838. For example, the implantable medical apparatus 800 can include a second electrode arrangement 830 interconnected via a lead body extension 860 to the dock 838 including the third electrode arrangement 840, which receives the first electrode arrangement 804. The dock 838 can include a recess/channel 848 extending through a first end 834A and opposite second end 834B of the dock 838. In one example, the elongate member 813 can be threaded through an opening 840A in the first end 834A of the dock 838 such that the elongate member 813 can be pulled (in the direction of the arrow from 834A to 834B) to guide the first electrode arrangement at least partially within the channel 832 of the dock to engage the first electrode arrangement 804 with the dock. In order to facilitate pulling the elongate member 813 into and through the channel 832 of the dock 838, in some examples, as shown in FIG. 5A, the second end 834B of the dock 838 may include a second opening 840B aligned with both the channel 848 and a central longitudinal axis (D) of the dock to permit feeding of the elongate member 813 into and through the dock 838. The opening 840B may, in some examples, include a seal at 842 to prevent fluid ingress between the elongated member 813 and the opening.

When the first electrode arrangement 804 is operatively engaged with the dock 838, the implantable medical apparatus 800 serves as an extension of the first electrode arrangement 804 when the second electrode 830 arrangement is operatively connected to a nerve.

In one example, the implantable medical apparatus 800 includes one or more anchors 870 positioned anywhere along a length of lead body extension 860. The anchor 870 can be configured to secure and maintain a position of the implantable medical apparatus 800 relative to tissue, such as non-nerve tissues. In some examples, the lead body extension 860 is flexible and/or made of a shape memory material so that an angle (Omega Ω) of the lead body extension with respect to a longitudinal axis of the dock (Omega Ω) can be varied prior to anchoring, if applicable. In one example, the lead body extension 860 extends from a longitudinal side/outer surface 839 of the dock 838, and at an angle (Alpha α) relative to a longitudinal axis of the dock (e.g., the angle being about 90 degrees +/−5 degrees, however, other angles are envisioned particularly in embodiments where the angle is variable/adjustable). In one example, the angle (Alpha α) is in a range of 10 to 170 degrees.

The implantable medical apparatus 800 of FIG. 5A can be used in many ways. Generally, in one example, the implantable medical apparatus 800 may be used to engage a target tissue/nerve T for stimulation or sensing via a first electrode arrangement. Engaging target tissue may, in some examples, include improving, increasing, and/or otherwise enhancing electrical capture of the target tissue or other forms of electrical communication relative to the target tissue. In various examples, the first electrode arrangement was previously chronically implanted and is not producing the desired stimulation or sensing due to inadequate positioning of the first electrode arrangement 804 with respect to the target nerve T.

In some example methods, an incision is formed in the patient and the implantable medical apparatus 800 is positioned adjacent to the target nerve T. In one example, the first electrode arrangement 804 may be linked via tether 813 at the time of the incision and, in other examples, the tether 813 may be threaded through the openings 840A, 840B post-incision (i.e. in vitro). In other examples, the tether 813 may be within the dock 838 during implantation of the implantable medical apparatus 800 and the tether 813 may be connected to the first electrode arrangement 804 from a location within the body in instances where the first electrode arrangement 804 was previously implanted. In either instance, the tether 813 is pulled via end 815 through the dock 383 until the first electrode arrangement 804 is engaged with the third electrode arrangement 840.

After the incision is formed, the second electrode arrangement 830 is positioned and operatively secured to the target nerve T and the first electrode arrangement 804 is engaged within the dock 838 (e.g. relative to the third electrode arrangement 840) according to examples of the disclosure. In essence, the implantable medical apparatus 800 forms a continuation of the first electrode arrangement 804 to engage the target nerve. In some examples, the implantable medical apparatus 800 will produce a better/stronger stimulation or sensing contact with the target tissue. In some methods, the lead body extension 860 can be manipulated and bent to achieve the desired positioning/angle of the second and/or third electrode arrangements 830, 840 with respect to the nerve, other patient anatomy, and first electrode arrangement 804, etc. Examples of the disclosure can include securing the implantable medical apparatus 800 in position with one or more anchors 870 at any time during the procedure, For example, anchoring may be done when the clinician feels confident that the implantable medical apparatus 800 is in the desired position and/or when securing the implantable medical apparatus 800 in position would enhance the ease of any remaining procedure steps. In one example, the angle (Omega Ω) is changed prior to the step of anchoring with anchor 870.

The second electrode arrangement 830 may, in some examples, be identical to electrode arrangement 630 of FIG. 3A. It is to be understood that the second electrode arrangement 830 may be alternatively configured according to any of the examples of the disclosure. In the examples of FIGS. 3A and 5, the second electrode arrangement 830 is illustrated as being a cuff including a tubular nonconductive carrier body 832 in which a plurality of electrodes 882 (e.g., stimulation electrodes), which may comprise ring electrodes or the like) are embedded.

An example of one such alternate configuration is shown in FIG. 5B. In this example, the second electrode arrangement 875 comprises a paddle configuration. In one example, the paddle configuration of the second electrode arrangement 875 can include a generally planar nonconductive carrier 884 that maintains and supports the plurality of electrodes (e.g., stimulation electrodes) The plurality of electrodes 882 may be arranged on or partially within the nonconductive carrier 884 in any fashion, as desired, such as being protruding from surface of carrier 884 or flush with the surface of the carrier 884 in some examples. In some examples, methods of use of an implantable medical apparatus disclosed above including the second electrode arrangement 875 of FIG. 5B would not differ from those disclosed above with the exception that the second electrode arrangement 875 would be positioned in relation to the target tissue in a manner corresponding to the configuration of the nonconductive carrier 884. In additional examples, any of the second electrode arrangements of the disclosure can include nonconductive carriers maintaining one or more cuff-shaped electrodes, ring-shaped electrodes or spit-ring-shaped electrodes or a combination thereof. Along these lines, it is reiterated that any of the electrode arrangements of the disclosure can include any of the disclosed alternate configurations.

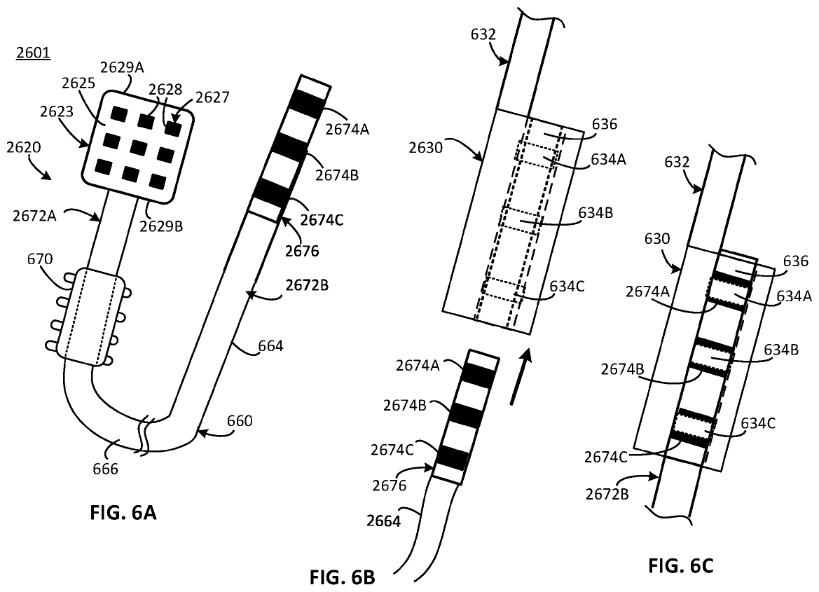

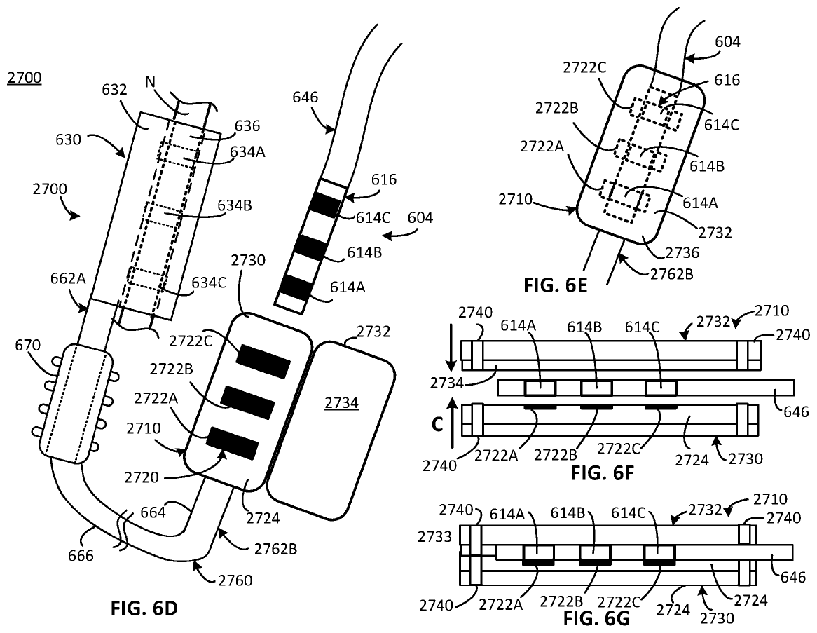

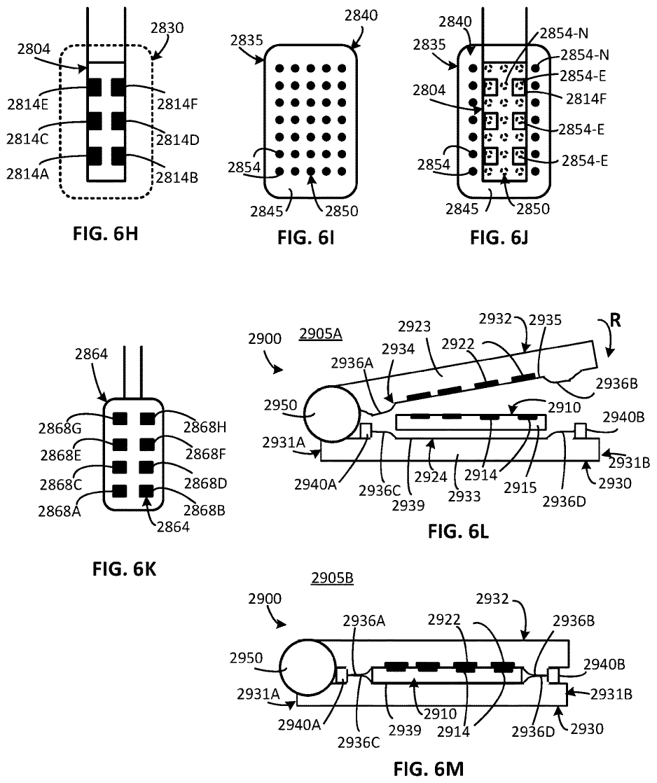

FIG. 6A-6C illustrate one example implantable medical apparatus 2601 for providing an alternate electrode arrangement to engage target tissue. In some examples, the apparatus 2601 comprises an example implementation of, and/or at least some of substantially the same features as, the example arrangements of FIGS. 1A-1B, 2A, 2B, 2C, 3A-3C, 5A-5B. However, unlike the example apparatus in FIGS. 3A, 3C, 5A, a second electrode arrangement 2623 comprises a paddle configuration (e.g. a generally planar non-conductive surface supporting an array of electrodes) instead of a cuff electrode configuration (e.g. 630 in FIGS. 3A, 3C; 830 in FIG. 5A).

It will be understood that some example configurations of the second electrode arrangement 2633 may comprise any symmetrically-shape carrier (e.g. electrically non-conductive surface/portion) supporting electrodes (e.g. non-ring electrodes in any one of various patterns, of which a paddle configuration may comprise just one example. In some such examples, at least the portion of the carrier supporting the electrode(s) (or supporting at least some of the electrode(s)) may comprise a generally planar surface portion. Of course, in some examples, at least a portion of the carrier also may comprise a non-symmetric shape.

With further reference to the example shown in FIG. 6A, in addition, unlike the example apparatus in FIGS. 3A-4, 5A, the third electrode arrangement 2676 in the example of FIGS. 6A-6C comprises an axial electrode arrangement (e.g. a cylindrically-shaped non-conductive carrier supporting ring or split-ring electrodes) instead of a dock-type electrode arrangement (e.g. 638, 738, 838 in FIGS. 3A-5A). With this in mind, FIG. 6B illustrates a lead 632 including a cuff electrode 2630 which has been previously implanted to engage a target nerve. In an example method, the cuff electrode 2630 is removed from the target nerve it was engaging, thereby making it available for engaging axial-style third electrode arrangement 2676 of apparatus 2601 (FIGS. 6A, 6B) in order to provide second electrode arrangement 2623 to engage a target nerve in a chronically implanted position to be in operative relation (e.g. stimulation and/or sensing) with the target tissue (e.g. nerve and/or muscle).

In particular, as shown in FIG. 6B, a longitudinal axis of the axial-style third electrode arrangement 2676 is aligned with a central longitudinal axis of lumen 636 of cuff electrode arrangement 2630 to permit insertion and advancement of third electrode arrangement 2676 into the lumen 636 until the electrodes 2674A, 2674B, 2674C of the axial-style third electrode arrangement 2676 become aligned with and electrically engage the corresponding electrodes 634A, 634B, 634C, respectively, of the cuff-style first electrode arrangement 2630. In some such examples, the cuff electrode 2630 may comprise one or more flaps (e.g. flanges extending from a base) which overlap each other to form lumen 636 and which are openable to provide side access to the lumen 636 (instead of a strictly end-access of lumen 636) to permit placement of axial-style third electrode within the lumen 636 after which such flaps are re-closed to removably secure the axial-style third electrode arrangement 2676 within the lumen 636.

In some examples, the axial-type electrode arrangement 2676 may comprise seals as in the example of FIG. 4 (e.g. seals 750A, 750B, 750C, 750D) or the cuff electrode 2630 (FIGS. 6B-6C) may comprise seals as in the examples of FIGS. 3A-3C (e.g. seals 650A, 650B, 650C).

Once this electrical connection is established between the electrodes 650A, 650B, 650C of the first electrode arrangement 2630 and the electrodes 2674A, 2674B, 2674C of the axial-style third electrode arrangement 2676, the paddle-style electrode arrangement 2623 is maneuvered into a desired position to in operative relation to a target tissue. In some examples, the paddle-style second electrode arrangement 2623 may first be maneuvered into operative relation to a target tissue prior to mating of the cuff-style first electrode arrangement 2630 and the axial-style third electrode arrangement 2673, as may dictated by which target tissue is being engaged, anatomical considerations, surgical preferences, etc.

FIG. 6D-6G illustrate one example implantable medical apparatus 2700 for providing an alternate electrode arrangement to engage target tissue. In some examples, the apparatus 2700 comprises an example implementation of, and/or at least some of substantially the same features as, the example arrangements of FIGS. 1A-1B, 2A, 2B, 2C, 3A-3C, and 5A-5B. For instance, in some examples, the second electrode arrangement 630 of apparatus 2700 in FIG. 6D may comprise a cuff electrode configuration as in FIGS. 3A, 3C (or 830 in FIG. 5A). In addition, unlike the example apparatus in FIGS. 3A-4, 5A, the third electrode arrangement in the example of FIGS. 6D-6G comprises a third electrode arrangement 2710 comprising a paddle-style electrode array 2720 and a cover (e.g., 2732 in FIG. 6D/6E; 2932 in FIGS. 6K-6L) instead of an axial-type electrode arrangement (e.g. 2676 in FIGS. 6A,6B) or instead of a dock-type electrode arrangement (e.g. 638, 738, 838 in FIGS. 3A-5A). With this in mind, FIG. 6D illustrates a lead 646 including an axial-style first electrode arrangement 604 which has been previously implanted to engage a target nerve. In an example method, the axial-style first electrode arrangement 604 is removed from the target tissue it was engaging, thereby making it available for engaging third electrode arrangement 2710 of apparatus 2700 in order to provide second electrode arrangement 630 (of 2700 in FIG. 6D) to engage a target nerve in a chronically implanted position to be in operative relation (e.g. stimulation and/or sensing) with the target tissue (e.g. nerve and/or muscle).

As further described below, in general terms the electrode arrangement 2710 including paddle-style base 2730 supporting electrode array 2720 is configured to receive a wide variety of differently sized, shaped, and/or oriented first electrode arrangements such as (but not limited to) axial-style arrangements (e.g. 604 in FIG. 6D), paddle-style arrangements, etc.

With this context in mind, as further shown in FIG. 6D, a longitudinal axis of the axial-style first electrode arrangement 604 is generally aligned with a longitudinal axis of paddle-style electrode array 2720 for placement thereon of axial-style first electrode arrangement 604 so that the electrodes 614A, 614B, 614C of the axial-style first electrode arrangement 604 become generally aligned with and electrically engage the corresponding electrodes 2722A, 2722B, 2722C of array 2720 in a one-to-one correspondence of the third electrode arrangement 2710, as further shown in FIG. 6E, to maintain individual addressability of each respective mated electrode pair 614A/2722A, 614B/2722B, 614C/2722C. In some such examples, the third electrode arrangement 2710 may comprise a cover 2732 which may be placed on top of the axial-style first electrode arrangement 604 and paddle electrode array 2720 (on base 2730) to sandwich the axial-style first electrode arrangement 604 between the elements 2730 and 2732 to sealingly contain the first electrode arrangement 604 therein. As later illustrated in the further examples of FIGS. 6F-6G or 6L-6M, the cover 2732 may be completely separate from base 2730 and secured onto base 2730 solely via fasteners (e.g. FIGS. 6F-6G) or the cover 2732 may be hingedly connected to the base 2730 (e.g. FIGS. 6L-6M) and the cover 2732 secured in a closed position via a fastener to compliment the hinged connection.

As further shown in FIG. 6D, the cover 2732 may comprise an inner surface 2734 for engaging the axial-style first electrode arrangement 604 and inner surface portion 2735 of base 2730 in order to sealingly contain the first electrode arrangement within the closed third electrode arrangement (e.g. closed arrangement of cover 2732 and base 2730, as further shown via the top view in FIG. 6E in which an outer surface portion 2736 of cover 2732 is seen with first electrode arrangement 604 (including electrodes 614A, 614B, 614C) engaging electrodes 2722A, 2722B, 2722C of base 2730, as represented via dashed lines.

Once this electrical connection is established between the electrodes 614A, 614B, 614C of the first electrode arrangement 604 and the electrodes 2722A, 2722B, 2722C of the axial-style third electrode arrangement 2710, the cuff electrode arrangement 630 (of 2700 in FIG. 6D) is maneuvered into a desired position to in operative relation to a target tissue. In some examples, the electrode arrangement 630 may first be maneuvered into operative relation to a target tissue prior to mating of the axial-style first electrode arrangement 604 and the third electrode arrangement 2710, as may dictated by which target tissue is being engaged, anatomical considerations, surgical preferences, etc.

FIGS. 6F-6G are each a sectional view representing an example implementation of third electrode arrangement in which the cover 2732 of comprises a component completely separable from the base 2730 with FIG. 6F representing an unassembled configuration and FIG. 6G representing an assembled configuration. In particular, as shown in FIG. 6F, with axial-style first electrode arrangement 604 positioned between base 2730 and cover 2732, apertures 2740 are aligned with each other, and upon application of a fastener(s) via the apertures 2740, the cover 2732 and base 2730 are drawn together as represented via directional force arrows C until an assembled configuration (i.e. assembled state) is achieved as shown in FIG. 6G. In the assembled configuration, the electrodes 2722A, 2722B, 2722C of base 2730 of third electrode arrangement 2710 pressingly engage the electrodes 614A, 614B, 614C of first electrode arrangement 604 while inner surface portions 2724, 2734 of the base 2730 and cover 2732, respectively, pressingly engage each other in a sealing manner to sealingly contain and isolate the interior of the assembled configuration from the surrounding environment. It will be understood that a wide variety of biocompatible materials and structures may be used to form the inner surface portions 2724, 2734 which cooperate to form and/or act as a seal. In some examples, at least a portion of one or both of the inner surface portions 2724, 2734 comprise a compressible material configured to conform about the axial-style electrode arrangement 604 in its sandwiched position between the base 2730 and cover 2732.

FIGS. 6H and 6K are each a top view of a first electrode arrangement including an alternate electrode configuration than in FIGS. 6D-6G. For instance, as shown in FIG. 6H, in some examples the first electrode arrangement 2800 comprises an axial-style electrode arrangement akin to the axial-style electrode arrangement 604 in FIGS. 6D-6G, except with the electrodes arranged as split-ring electrodes 2814A-2814F instead of ring electrodes 614A, 614B, 614C (FIG. 6D). In some examples, each split ring electrode 2814A-2814F is individually addressable (e.g. controllable). Accordingly, in some such examples, in order to maintain such individual addressability, the electrode configuration of the third electrode arrangement to mate with the electrodes 2814A-2814F of first electrode arrangement may comprise a configuration other than that shown in FIG. 6D (e.g. 3 planar electrodes on base 2730), such as the third electrode arrangement comprising the configuration shown in FIG. 6I. As shown in FIG. 6I, example third electrode arrangement 2835 comprises a base 2840 (e.g. akin to base 2730 in FIG. 6D) including an array 2850 of electrodes 2854 on non-conductive portion 2845. The array 2850 comprises a quantity (e.g. 30) of electrodes 2854 which exceeds a quantity (e.g. 8) of electrodes 2814A-2814F of the first electrode arrangement, and with electrodes 2854 arranged in a spaced apart pattern (e.g. rows and columns or other patterns) such that at least some of the electrodes 2854 of array 2850 have a position matching one of the electrodes 2814A-2814F, as further shown in FIG. 6J in which the electrodes 2814A-2814F of the first electrode arrangement 2804 generally overlies the electrode array 2850 (of base 2830) of third electrode arrangement 2835. For instance, as shown in FIG. 6J, electrode 2814F overlies and electrically engages electrode 2854-E (E=engage) while electrode 2814F does not overlie/engage various electrodes 2854-N (N=not engage). In some examples, the quantity of electrodes 2854 of third electrode arrangement 2835 exceeds the quantity of electrodes of the first electrode arrangement by a factor of at least 1.10, 1.25, 1.5, 1.75, or 2, and in such examples, the quantity of electrodes 2854 may sometimes be referred to as substantially exceeding the quantity of electrodes 2814A-2814F of the first electrode arrangement.

Via this arrangement, regardless of the quantity, size, shape, and/or position of the electrodes of the first electrode arrangement (e.g. 2804), the third electrode arrangement provides a format (e.g. quantity, size, shape, position) of electrodes by which each electrode of the first electrode arrangement (e.g. 2804) will uniquely electrically engage at least one electrode of the third electrode arrangement. Via this arrangement, the third electrode arrangement is capable of electrically mating with a wide variety of first electrode arrangements while maintaining individual addressability of the electrodes of the first electrode arrangement.

FIG. 6K is a top plan view representing an example first electrode arrangement in a paddle-style arrangement including an array 2864 of electrodes 2868A-2868H, which may be suitable for engagement by the array 2850 (FIG. 6I) of electrodes 2854 of third electrode arrangement 2840 which are sized, shaped, positioned, and comprise a quantity to uniquely mate with electrodes 2868A-2868H in order to achieve/maintain individual addressability of the electrodes 2868A-2868H.

FIGS. 6L-6M each are sectional views of an example third electrode arrangement 2900 including a base 2930 and cover 2932 in a manner akin to the third electrode arrangement of FIGS. 6D-6E, and 6F-6G, except with the third electrode arrangement 2900 including a hinged connection 2950 between its base 2930 and cover 2932 to enable manipulating the third electrode arrangement 2900 between an open configuration 2905A (i.e. open state) shown in FIG. 6L and a closed configuration 2905B (i.e. closed state) shown in FIG. 6M.

As shown in FIG. 6L, the cover 2932 includes inner surface portion 2934, which comprises electrodes 2922 on a first surface portion 2935, which may be generally planar in some examples, and which comprises raised second surface portion 2936A, 2936B on opposite ends and sides of the first surface portion 2935 (and therefore on opposite sides and ends of the electrodes 2922).

The entire third electrode arrangement 2900 comprises a first end portion 2931A and opposite second end portion 2931B, with hinged connection 2950 at the first end portion 2931A to pivotally connect cover 2932 to base 2930. As further shown in FIG. 6L, the base comprises inner surface portion 2924, which omits electrodes on a first surface portion 2939, which may be generally planar in some examples, and which comprises raised second surface portion 2936C, 2936D on opposite ends and sides of the first surface portion 2924 (and therefore on opposite sides and ends of the inner surface portion 2924). The inner surface portion 2924 may be sized and shaped to removably receive at least a portion of the first electrode arrangement 2910, and to position the first electrode arrangement 2910 so that electrodes 2914 will face and match a corresponding electrodes 2922 of the inner surface portion 2934 of cover 2930 of the third electrode arrangement 2900. In some examples, the first electrode arrangement 2910 may take the form of a paddle-style arrangement (e.g. FIG. 6K), but may take other forms such as axial-style electrode arrangement (e.g. FIG. 6H) with electrodes 2922 of third electrode arrangement 2900 being sized, shaped, and/or having a quantity suitable for mating with the electrodes of the axial-style first electrode arrangement.

With further reference to FIG. 6L, in one example method, with first electrode arrangement 2900 in its open state 2905A, the first electrode arrangement 2904 is positioned into the space between the separated cover 2932 and base 2930. Thereafter, as represented via directional force arrow R, the cover 2930 and base 2930 are pivotally moved toward each other as supported and guided via hinged connection 2950 until the cover 2932 meets and mates with the base 2930 as shown in FIG. 6M to achieve the closed configuration 2905B. As further shown in FIG. 6M, the raised second inner surface portion 2936A 2936B of cover 2932 pressingly engages the raised second inner surface portion 2936C, 2936D of base 2930, such that the respective surface portions 2936A, 2936B, 2936C, 2936D may at least partially compress each other to act and form a seal which sealingly contains the first electrode arrangement 2910 within an interior of the closed configuration of the cover 2932 and base 2930.

In addition to, instead of, and/or as part of the inner surface portion 2936C, 2936D, at least seal 2940A, 2940B acts to sealingly contain the first electrode arrangement within the closed configuration of the cover 2932 and base 2930 of the third electrode arrangement 2900.

It will be understood that the electrodes 2914 of the first electrode arrangement 2910 shown in FIG. 6L-6M may represent either an array with a size, shape, and quantity that generally corresponds to (i.e. a one-to-one match) with the electrodes 2922 of the cover 2930 of third electrode arrangement 2900 or may represent a size, shape, position, and quantity akin to the example of FIG. 6J in which the quantity of electrodes 2922 exceed the quantity of electrodes 2914, so that just some of electrodes 2922 electrically engage the electrodes 2914 of the first electrode arrangement.

In some examples, the electrodes 2922 may be present on the inner surface portion 2939 of the base 2930 instead of being present on cover 2930 or electrodes 2922 may be present on inner surface portion 2934, 2924 of both of the cover 2932 and base 2930, respectively.

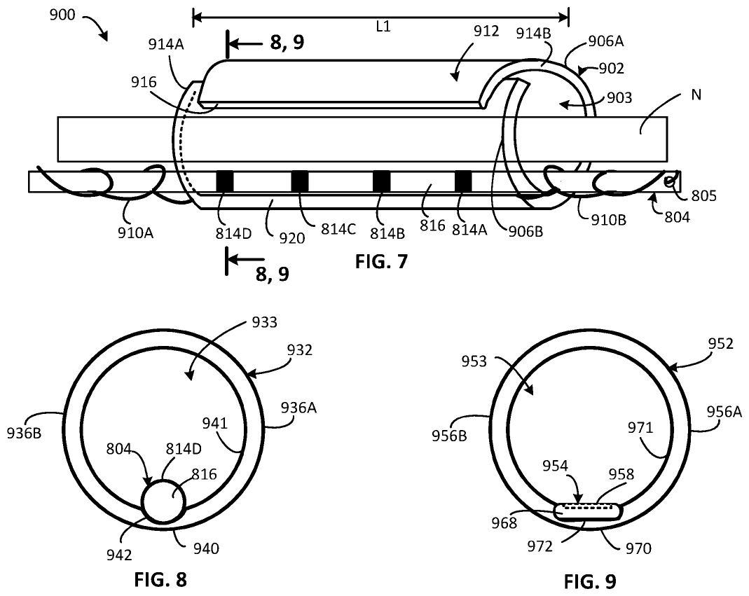

FIG. 7 illustrates an example of an implantable medical apparatus 900 that can be configured to engage a target nerve by a first electrode arrangement 804. In various examples, the implantable medical apparatus 900 includes a wrap 902 (shown as transparent for ease of illustration) positioned around a circumference of at least one of the first electrode arrangement 804 and the nerve N to urge, in close proximity, the first electrode arrangement 804 against the nerve, engaging the nerve N. In one aspect, this arrangement may increase a degree of electrical capture (e.g. for stimulation) or other electrical communication (e.g. for sensing) of the first electrode arrangement 804 relative to the nerve N. In some examples, the increased degree of electrical capture may comprise and/or be exhibited as an increased response of the targeted nerve and/or targeted muscle to the delivered stimulation. In some examples, the increased degree of electrical capture may comprise and/or be exhibited as achieving a suprathreshold level of stimulation (of the target tissue) but at energy levels (e.g. voltage amplitude) which are lower than energy levels previously delivered via the first electrode arrangement to achieve a suprathreshold level of stimulation.

Therefore, it is envisioned that the wrap 902 can be made of various sizes in both diameter and length based on the desired surgical application. In one example, the wrap 902 has a length L1 that is sufficient to cover/sheathe the electrodes 814A-814D of the first electrode arrangement 804. In one example, the wrap 902 is made of a nonconductive material (i.e. the wrap is nonconductive in its entirety, in some examples). According to some examples of the disclosure, the wrap 902 is a singular, uniform piece of material that can define a lumen 903 in a natural (unbiased) arrangement. In some examples of the disclosure, the wrap 902 may include sections having differing configurations. In one example, the wrap 902 may be viewed as having two interconnected arms 906A, 906B that can furl to overlap each other from an imaginary boundary of the wrap, opposite an overlapping portion 912. In various examples, the wrap 902 can be viewed as comprising a furled body having a free end 916 and is configured to have the overlapping portion 912 when operatively positioned around at least one of the first electrode arrangement 804 and the nerve N as is shown in FIG. 7. In one example, the arms 906A, 906B may be configured to face each other in opposite directions and may be biased in the furled, closed configuration but may be temporarily spread apart to enable fitting the wrap 902 about the nerve N. In some examples, the arms may extend in the same orientation, spaced along a length of the wrap.

In one example, the wrap 902 has a natural arrangement (i.e. an arrangement free of external forces) in which the wrap 902 spans less than 360 degrees (i.e. the wrap does not fully close in the natural arrangement). In one example, the wrap 902 has a natural arrangement (i.e. an arrangement free of external forces) in which the wrap spans at least 360 degrees in its closed position. In one example, the wrap 902 has a natural arrangement (i.e. an arrangement free of external forces) in which the wrap 902 overlaps itself to span more than 360 degrees in its closed position/arrangement. In such embodiments, the wrap 902 may be biased into the curled, natural arrangement. Various examples of the wrap 902 may comprise a single member or sheet that is biased to furl and/or encircle another structure. In various methods, each wrap 902 can be unfurled so that it can be wrapped around at least one of a first electrode arrangement 904 and a nerve N once released. To maintain the implantable medical apparatus 900 in position once the wrap 902 is deployed around the nerve N and/or first electrode arrangement 804, various examples can include at least one tether 910A, 910B, such as a flexible, filament, wire, suture, extension of the wrap material or similar material. In one example, a tether 910A, 910B is provided at each longitudinal end 914A, 914B of the wrap 902, for example. Each tether 910A, 910B can be permanently attached to the wrap in one example of the disclosure. Each tether may be used to secure the wrap 902 in longitudinal position to the first electrode arrangement 804 in one of many ways, which can include tying or winding the tether 910A, 910B around the first electrode arrangement 804 and relying on friction to hold the tether 910A, 910B in position with respect to the first electrode arrangement. Such an attachment method could be considered a passive method of attachment. In some examples, active methods of attachment, such as fixating the tether 910A, 910B to the first electrode arrangement 804 with sutures or other mechanical means, may be employed. In some such examples, the first electrode arrangement 804 may comprise at least one aperture (e.g. suture hole) to facilitate securing the tether 910A, 910B and/or sutures to electrically non-conductive carrier portion 816 of the first electrode arrangement 804. In various examples, the overlapping portion 912 may be configured to maintain a diameter of the wrap 902 once deployed. For example, the overlapping portion 912 can include adhesive or the like. In some examples, a suture or the like may be tied around an outer circumference of the wrap, similar to a belt, to maintain the respective wrap in position.

In this example, the first electrode arrangement 804 is the same as previously disclosed electrode arrangement 804, however, the first electrode arrangement can take other configurations. It is to be understood that at least some, or all, other aspects of the implantable medical apparatus of FIG. 7 can be similar or identical to that of FIGS. 1A-6 and that other features of other embodiments disclosed herein may also be incorporated into the embodiment of FIG. 7.

Via the example arrangement in FIG. 7, the tether 910A, 910B and size/shape of the wrap 902 act together to closely secure the wrap 902 relative to the first electrode arrangement 804, while the arms 906A, 906B of the wrap 902 act to closely secure the wrap 902 about the nerve N, which in turn urges and maintains the first electrode arrangement 804 in close proximity to the nerve N.

FIG. 8 is a sectional view illustrating an example wrap 932. It is to be understood that at least some, or all, other aspects of the wrap 932 of FIG. 8 can be similar or identical to that of FIGS. 1A-7 and that other features of other embodiments disclosed herein may also be incorporated into the embodiment of FIG. 8. In this example, the wrap 932 includes a base region 940. In various examples, the base region 940 will, at least in part, interconnect two arms 936A, 936B (or additional arms in examples where multiple arms are interconnected by a base on each side of the base). In some of these examples, the base region 940 and/or arms 936A, 936B are configured to cause arms 936A, 936B to be biased into a closed position, as in the example of FIG. 8 or other configurations. In one example, the base region 940 has an interior surface 941 defining a groove 942 that corresponds to a configuration of the carrier body of the first electrode arrangement (e.g., carrier body 816 of electrode arrangement 804) such that the first electrode arrangement can be received within the groove 942. In one example, the groove 942 spans a full longitudinal length of the wrap (see also, longitudinal length L in FIG. 7). The corresponding configuration of the groove 942 may correspond to at least one of the size of and/or cross-sectional shape of the carrier body 816/electrode arrangement 804.

The example shown in the sectional view of FIG. 9 illustrates an example wrap 952 having an inner surface 971 defining an alternately configured groove 972 at base region 970. In some examples, similar to that of FIG. 8, the groove 972 corresponds to the configuration (e.g. shape, size) of the carrier body of a first electrode arrangement 954 via the groove 972 comprising a cross-sectional size or area that is less than a cross-sectional size or area of the carrier body of the first electrode arrangement. Accordingly, in some examples, when viewed in cross-section, the groove 972 may be referred to as partially surrounding (i.e. encircling) the carrier body 968 of the first electrode arrangement 954 such that electrode(s) 958 of the first electrode arrangement 954 are exposed within a lumen 953 formed by the inner surface 971 of the wrap 952 to engage the first electrode arrangement to be in stimulating relation and/or sensing relation to the target tissue (e.g., nerve).

It is to be understood that at least some, or all, other aspects of the wraps of FIGS. 8-9 can be similar or identical to that of FIGS. 1A-7 and that other features of other embodiments disclosed herein may also be incorporated into the embodiments of FIGS. 8-9. For example, it will be understood that FIGS. 8 and 9 provide a simplified view generalizing the manner in which the respective separate arms (e.g. 936A, 936B in FIG. 8; 956A, 956B in FIG. 9) overlap and/or otherwise engage each other.

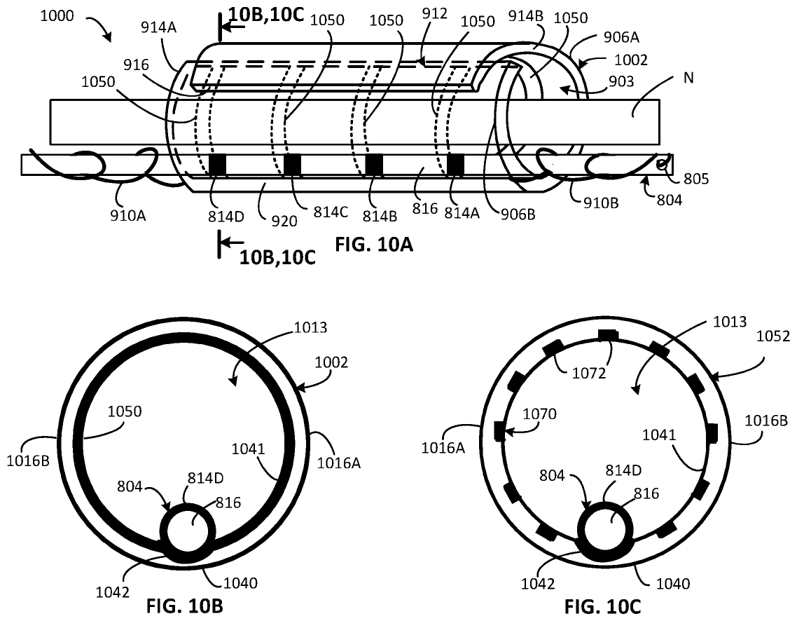

FIGS. 10A-10C are diagrams illustrating example wraps 1002, 1052 which comprise at least some of substantially the same features as the wraps 902, 932, 952 of FIGS. 7-9, except with wraps 1002, 1052 additionally comprising their own electrodes to engage a target nerve. In particular, FIG. 10A is a front view of an example arrangement 1000 including a wrap 1002 like wrap 902 (FIG. 7), except additionally comprising electrodes 1050 (shown in dashed lines) extending at least partially circumferentially about an interior surface of the wrap 1002 and exposed to face the target nerve N. In some examples, each electrode 1050 may comprise a ring-shaped conductive element having a width similar to a width of each electrode 814A, 814B, 814C, 814D (e.g. ring electrode) of the first electrode arrangement 804. Upon placement of the wrap 1002 about the first electrode arrangement 804 and nerve N, the electrodes 1050 pressingly engage the electrodes 814A-814D, respectively, to establish electrical connection therebetween, and the electrodes 1050 pressingly engage the nerve N to cause the electrodes 1050 to be in operative relation (e.g. stimulating relation and/or sensing relation) to the nerve N. Because the respective electrodes 1050 are spaced apart from each other along a length of the wrap 1002, they are individually addressable at least to the extent that electrodes 814A, 814B, 814C, 814D (to which the electrodes 1050 are in electrical engagement) are individually addressable. Among other aspects, the electrodes 1050 may substantially increase the surface area of electrodes available to contact an outer surface of the nerve. It will be understood that surface area of electrode 1050 may have a value to achieve the desired electrical parameters of electrical communication between each electrode 1050 and nerve N. Accordingly, in some examples, each electrode 1050 may extend around a full circumference of inner surface of the wrap or just a portion of the full circumference, such as a half-ring, quarter ring size, etc.

FIG. 10B is a sectional view, as taken along lines 10B-10B of FIG. 10A, further illustrating one electrode 1050 extending about a circumference of inner surface 1041 of wrap 1002. In some examples, the wrap 1002 comprises at least some of substantially the same features as the example wrap in FIG. 8, except with the addition of electrode 1050. Accordingly, wrap 1002 includes base region 1040 (like base 940), arms 1016A, 1016B (like arms 936A, 936B), interior surface 1041 (like 941). In some examples, the wrap 1002 may additionally comprise groove 1042 configured to receive electrode arrangement 804. In one example, at each position along a length of the wrap which corresponds to the location of each electrode 1050, the groove 142 comprises an electrically conductive portion which is sized and shaped to establish electrical communication between the electrode 814D of first electrode arrangement 804 and the electrode 1050.

FIG. 10C is a sectional view like the sectional view of FIG. 10B illustrating a wrap 1052 comprising at least some of substantially the same features as wrap 1002 of FIGS. 10A, 10B, except with each electrode 1050 being replaced by a ring of spaced apart electrode portions 1072 which may function together as a single electrode 1070 (like 1050). Among other aspects, the spaced part electrode portions 1072 may enhance flexibility of the wrap 1052 and decrease the amount of electrically conductive material being used while still retaining a relative high degree of conductive material in contact with the nerve N. It will be understood that the electrode portions 1072 may be in electrical communication with each other and with electrically conductive portion of groove 1042 via conductor(s) extending within the wrap 1052.

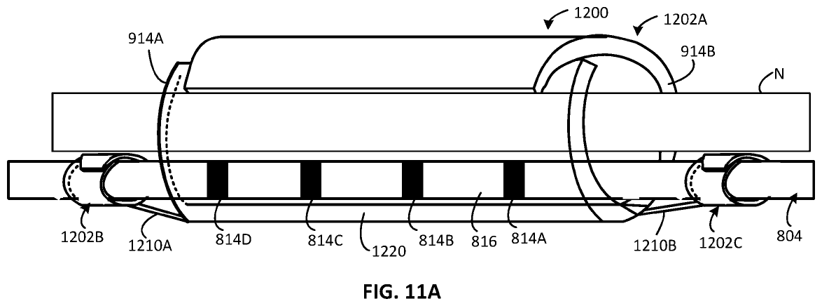

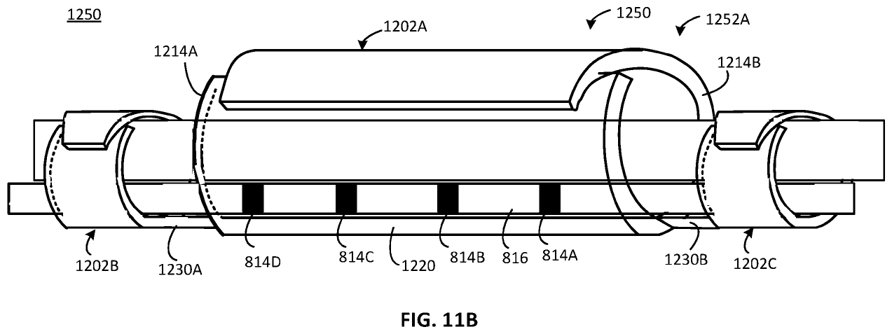

FIG. 11A illustrates an example implantable medical apparatus 1200. In some examples, apparatus 1200 may comprise at least some of substantially the same features as the examples of at least FIGS. 7-10C, while further comprising additional features such as wraps 1202B, 1202C. In various examples, the implantable medical apparatus 1200 includes a plurality of wraps 1202A (shown as transparent for ease of illustration), 1202B, 1202C positioned around a circumference of at least one of the first electrode arrangement 804 and the nerve N to urge and maintain, in close proximity, the first electrode arrangement 804 against the nerve, achieving electrical capture of the nerve N. In one example, a first or main wrap 1202A is positioned around the electrodes 814A-814D of the first electrode arrangement 804 and the nerve N and two second/third side wraps 1202B, 1202C are positioned on opposite ends of the main wrap 1202A and encircle the first electrode arrangement 804 (and not the nerve N). In some examples, the second and third wraps 1202B, 1202C are spaced apart from a respective opposite ends of the first wrap 1020A. In the illustrated example, the side wraps 1202B, 1202C are interconnected to the main wrap 1202A with tethers 1210A, 1201B or the like and maintain a longitudinal position of the main wrap 1202A with respect to the nerve N and the first electrode arrangement 804. The tethers 1210A, 1210B can be flexible or rigid.

FIG. 11B illustrates the implantable medical apparatus 1200 of FIG. 11A in an example arrangement 1250 in which the plurality of wraps 1202A, 1202B, 1202C are positioned around a circumference of both the first electrode arrangement 804 and the nerve N to urge and maintain, in close proximity, the first electrode arrangement 804 against the nerve N to increase and/or maintain a degree of electrical capture of the nerve N. In one example, one wrap 1202B is on a first end 1214A of wrap 1202A and wrap 1202C is on a second end 1214B of the wrap 1202A. In one example, side wraps 1202B, 1202C are each connected to wrap 1202A via an extension portion 1230A, 1230B, which may be a continuation of the material of the wrap 1202A. In one example, the extension portion 1230A, 1230B is made of a semi-rigid material. In various examples, one or more extension portions 1230A may be a tether, suture, wire, filament or other elongated member that may or may not be flexible.

In the example of FIGS. 11A-11B, the first electrode arrangement 804 is the same as previously disclosed electrode arrangement 804, however, the first electrode arrangement can take other configurations. It is to be understood that the side wraps of FIGS. 11A-11B can have any of the properties of the other wraps of the disclosure. It is to be further understood that at least some or all other aspects of the implantable medical apparatus of FIGS. 11A-11B can be similar or identical to that of FIGS. 1A-10C and that other features of other embodiments disclosed herein may also be incorporated into the embodiments of FIGS. 11A-11B.

In some examples, the apparatuses of FIGS. 7A-11B sheathe the first electrode arrangement and urge the first electrode arrangement against the target nerve to electrically engage the first electrode arrangement with the target nerve. In some examples, additional elements may be utilized to maintain the alignment and positioning of aspects of the apparatus so that the first electrode arrangement remains both sheathed and engaged with the first electrode arrangement. In some examples, the implantable medical apparatus is configured such that it can be implanted in a second surgical procedure after it is determined that the first electrode arrangement alone is insufficient to engage the target nerve.

Example methods of using the implantable medical apparatuses of FIGS. 7-11B may include the following. In one example, the first electrode arrangement 804 is positioned adjacent to a target nerve or tissue N. In various examples, the step of positioning may be done in a first surgical procedure that is later deemed ineffective or otherwise insufficient for stimulation or sensing due to the positioning of the first electrode arrangement. The first surgical procedure may or may not have an incision or direct visualization of the target nerve. In some examples, the implantable medical apparatus (e.g., 900, 1100, 1200) is unfurled adjacent the first electrode assembly so that the free edge (e.g., free edge 916) no longer overlaps and the wrap is positioned around the first electrode arrangement, within the lumen formed by the furled wrap in the natural arrangement. In some examples, the nerve or target tissue will additionally be positioned within the lumen. Then, the wrap may be released so that it automatically transitions to its biased natural arrangement in which the first electrode arrangement is urged into close proximity and maintained relative to (e.g. against) the nerve or target tissue to achieve a desired degree of electrical capture according to examples of the disclosure. In various examples including one or more side wraps (e.g., 1202B, 1202C), the one or more side wraps can similarly be secured over the nerve and/or first electrode arrangement and/or nerve. Various embodiments may include activating, accessing or applying, etcetera, a fastening mechanism, such as adhesive, to secure the flap (e.g., 912) and/or free edge (e.g., 916) in position, thus fixing a diameter of the lumen of the respective wrap.

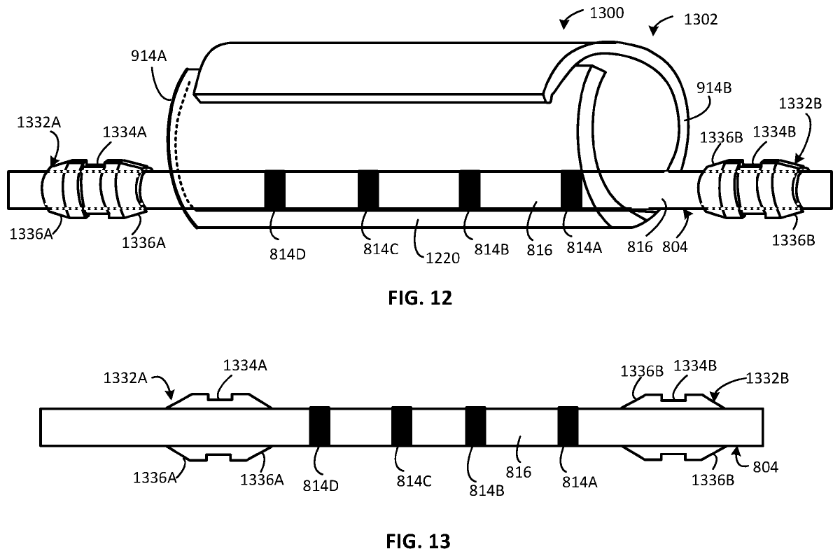

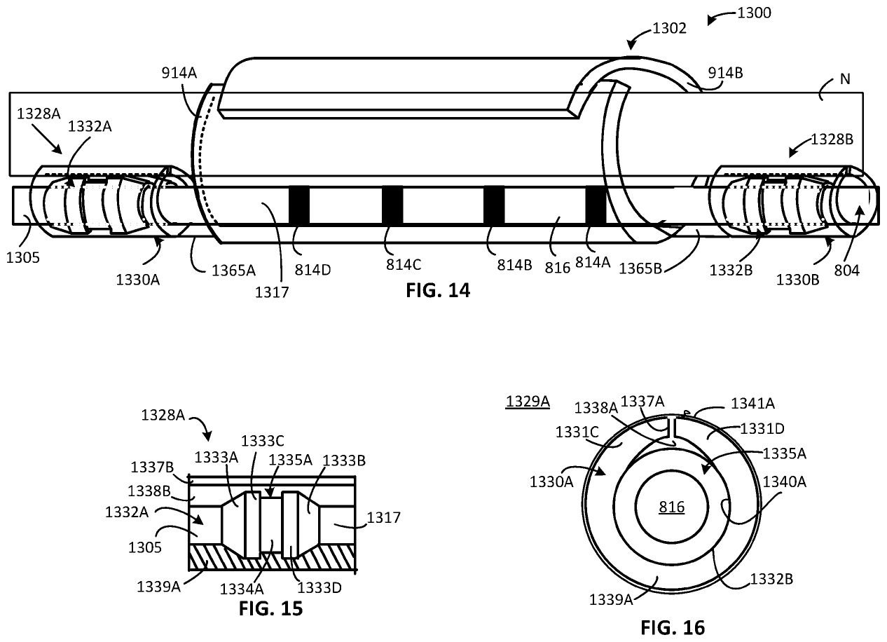

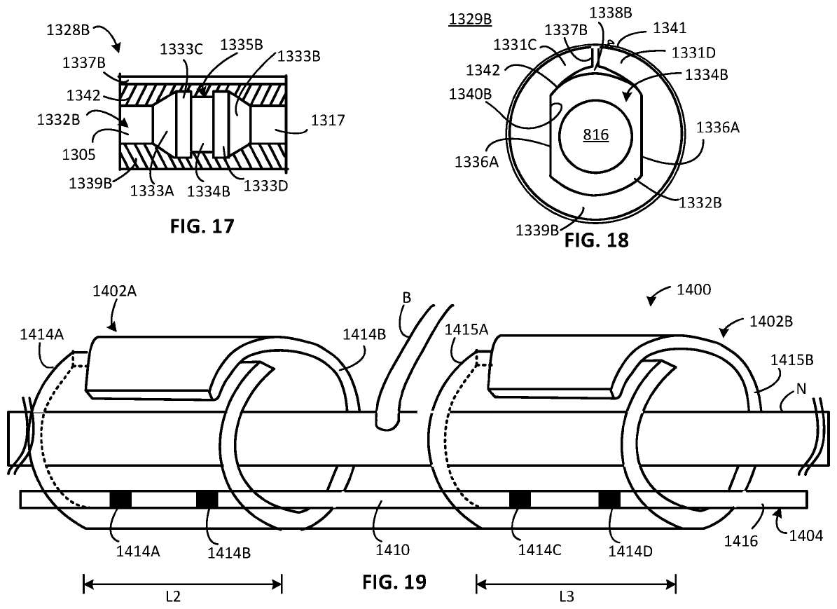

FIGS. 12-14 collectively illustrate another example of an implantable medical apparatus 1300. It is to be understood that at least some, or all, aspects of the implantable medical apparatus 1300 of FIGS. 12-14 can be similar or identical to that of FIGS. 1A-11B and that other features of other embodiments disclosed herein may also be incorporated into the embodiment of FIGS. 12-14. In this example, the implantable medical apparatus 1300 includes a wrap 1302 (shown as transparent for ease of illustration) that can include any properties of any wrap disclosed herein. As shown in FIG. 14, the implantable medical apparatus 1300 further includes at least one clip 1330A, 1330B (each shown as transparent for ease of illustration). Each clip 1330A, 1330B can be rigid or non-rigid. In the illustrated example, one clip 1330A, 1330B is provided on each end of the wrap 1302 so that the wrap 1302 is interposed between, and spaced apart from, the respective clips 1330A, 1330B. As shown in at least FIGS. 12-13, in one example, at least one retainer 1332A, 1332B is fixedly secured to the nonconductive carrier 816 of the first electrode arrangement 804 so that it is immovable longitudinally and/or rotationally with respect to the first electrode arrangement 804. Collectively, the clip 1330A, 1330B and retainer 1332A, 1332B can form an assembly 1328A, 1328B (see also, FIGS. 15 and 17, respectively). Each retainer 1332A, 1332B is configured to receive one clip 1330A, 1330B as is shown in FIG. 14, for example. In one example, each retainer 1332A, 1332B defines a circumferential groove 1335A, 1335B disposed between two tapered ends 1333A, 1333B. According to one example of the disclosure, at least one clip 1330A, 1330B can be used to secure the wrap 1302 in position about the first electrode arrangement 804 by positioning the clip 1330A, 1330B, which is interconnected to the wrap 1302 with an extension 1365A, 1365B or the like, onto one respective retainer 1332A, 1332B (FIG. 14). In one example, one or more clips 1330A, 1330B has separate arms that can open and close and form a shell to grasp one respective retainer 1332A, 1332B. The clips 1330A, 1330B may alternatively be referred to as an anchor in which sutures can be tied to secure the wrap 1302 to the anchors without damaging the underlying structure of the first electrode arrangement.

In some examples, the wrap 1302 can include any properties of any other wraps disclosed herein, such as wrap 902, for example. In the example of FIGS. 12-14, the first electrode arrangement 804 is the same as previously disclosed electrode arrangement 804, however, the first electrode arrangement can take other configurations. It is to be understood that at least some, or all, other aspects of the implantable medical apparatus of FIGS. 12-14 can be similar or identical to that of FIGS. 1A-13 and that other features of other embodiments disclosed herein may also be incorporated into the embodiment of FIGS. 12-14.

Methods of using the implantable medical apparatus of FIGS. 12-14 can include any of the steps disclosed with respect to the examples of FIGS. 7-11 and can further include a step of securing each clip 1330A, 1330B to the respective retainer 1332A, 1332B of the first electrode arrangement 804. One or more clips 1330A, 1330B may be secured to the respective retainer 1332A, 1332B either before or after the wrap 1302 is positioned around the target nerve N and/or first electrode arrangement 804.

FIGS. 15-18 collectively illustrate how, in some examples, a shape of the retainer 1332A, 1332B of FIGS. 12-14 can correspond to a shape of the corresponding clip 1330A, 1330B so that the clip can be connected to the retainer passively based on interlocking geometry. In FIGS. 15 and 16, for example, at least one retainer 1332A can include at least one tapered portion 1333A, 1333B. In various examples, one tapered portion 1333A tapers away from a first portion 1305 of the carrier body 816 that is free from electrodes and the other tapered portion 1333B tapers away from a second portion 1317 of the electrode arrangement 804. In one example, the second portion 1317 maintains electrodes 814A-814D. In one example, each tapered portion 1333A, 1333B is connected to a side portion 1333C, 1333D on each end of an interconnecting portion 1334A. In one example, the side portions 1333C, 1333D may be cylindrical in shape but other shapes are envisioned. Collectively, the portions 1333C, 1333D and 1334A define a circumferential groove 1335A around the interconnecting portion 1334A. In one example, the interconnecting portion 1334A and groove 1335A can be configured to have a circular cross-section to correspond to a generally circular or circular cross-section of at least part of an inside surface 1340A of the clip 1330A. In one example, the interconnecting portion 1334A has a circumference that is smaller than a circumference of each of the side portions 1333C, 1333D so that the interconnecting portion 1334A is recessed with respect to the side portions 1333C, 1333D. Taken together, these shapes form corresponding geometry that facilitates secure engagement of the clip to the retainer in an interlocking fashion.

In some examples, the clip 1130A includes first and second arms 1331A, 1331B that define a gap at 1337A that can extend a length of the clip 1330A. The arms 1331A, 1331B may be resilient in some examples so that they may flex with respect to each other, to enlarge the gap 1337A so that the clip 1330A may be positioned around the retainer 1332A. In some examples, as shown in an arrangement 1329A of FIG. 16, an open space 1338A may be formed between the gap 1337A and the interconnecting portion 1334A. Opposite the gap 1337A, the arms 1331A, 1331B are joined at a base region 1339A. In some examples, a suture 1341 can be used to further affix the clip 1330A to the respective retainer 1332A to restrict longitudinal and rotational movement of the clip with respect to the retainer. It some examples, retainer 1332B is identically configured to retainer 1330A and clip 1330B is identically configured to clip 1330A. In other examples, the retainers and or clips may differ in construction.

Additional interlocking configurations for one or more of the clips 1330A, 1330B and retainers 1332A, 1332B are envisioned. In FIGS. 17 and 18, for example, an interconnecting portion 1334B may be configured to have a cross-section having straight sides 1336A, 1336B which may correspond to a shape of an internal surface 1340B of alternate clip 1330B as shown in the arrangement 1329B (FIG. 18). Collectively, the portions 1333C, 1333D and 1334B define a circumferential groove 1335B around the interconnecting portion 1334B. In one example, the interconnecting portion 1334B and groove 1335B are configured to have another non-circular cross-section to correspond to a cross-section of at least part of the inside surface 1340B of the clip 1330B. In one example, the interconnecting portion 1334B and groove 1335B may form an oval cross-section having straight sides 1336A. In one example, the interconnecting portion 1334B has a circumference that is smaller than a circumference of each of the side portions 1333C, 1333D so that the interconnecting portion 1334A is recessed with respect to the side portions 1333C, 1333D.

In some examples, the clip 1330B includes first and second arms 1331C, 1331D that define a gap at 1337B that can extend a length of the clip 1330B. The arms 1331C, 1332D may be resilient in some examples so that they may flex with respect to each other, to enlarge the gap 1337B so that the clip 1330B may be positioned around the respective retainer 1332B. In some examples, as shown in an arrangement 1329B of FIG. 18, an open space 1338B may be formed between the gap 1337B and the interconnecting portion 1334B. As shown in FIG. 18, the open space 1338B may be much smaller as compared to that of FIG. 16 due to the more elongated, oval-esque shape of the interconnecting portion 1334B extending toward gap 1337B at 1342. Opposite the gap 1337B, the arms 1331C, 1331D are joined at a base region 1339B. In some examples, suture 1341 can be used to further affix the clip 1330B to the respective retainer 1332B to restrict longitudinal and rotational movement of the clip with respect to the retainer. It some examples, retainer 1332B is identically configured to retainer 1332A and clip 1330B is identically configured to clip 1330A. In other examples, the retainers and or clips may differ in construction.

FIG. 19 schematically illustrates an implantable medical apparatus 1400 according to another example of the disclosure. It is to be understood that at least some, or all, other aspects of the implantable medical apparatus 1400 of FIG. 19 can be similar or identical to that of FIGS. 1A-18 and that other features of other embodiments disclosed herein may also be incorporated into the embodiment of FIG. 19. In one example, the implantable medical apparatus 1400 includes two wraps 1402A, 1402B arranged side-by-side in series and interconnected with at least one spacer 1410 (or any tether of the disclosure). In one example, the spacer 1410 is rigid or semi-rigid and maintains a fixed distance between the wraps 1402A, 1402B. In other various examples, the spacer 1410 is made of a flexible, resilient material. In one example, the spacer 1410 is made of a non-conductive material and does not include electrically conductive material. Each wrap 1402A, 1402B can be of any of the types disclosed herein such as wrap 902, for example. Furthermore, each wrap 1402A, 1402B may be identically configured or may be differently configured according to various examples of the disclosure.

In various methods, each wrap 1402A, 1402B can be unfurled so that it can be wrapped around at least one of a first electrode arrangement 1404 and a nerve N. In one example, one wrap 1402A may be positioned on one side of a branch B of the nerve N and another wrap 1402B may be positioned on an opposite side of the branch B. To further accommodate such positioning and engagement with the nerve N, the first electrode arrangement of this example may have a plurality of electrodes 1414A, 1414B, 1414C, 1414D that are unevenly spaced along a length of a carrier body 816 supporting the electrodes. However, in some examples, the electrodes 1414A, 1414B, 1414C, 1414D may be evenly spaced apart along carrier body 816.

In one example, the wraps 1402A, 1402B may be positioned as to collectively cover all of the plurality of electrodes 1414A-1414D, while urging the respective plurality of electrodes into engaging relation with the nerve N, such as for stimulation and/or sensing. Additional method steps and features disclosed with respect to other embodiments including one or more wraps can additionally be utilized. For example, it is envisioned that the implantable medical apparatus 1400 can include one or more wraps that are of different shapes/sizes to best suit the target tissue and other implant objectives. It is further envisioned that, in some examples, one or more wraps may be wrapped around the first electrode arrangement but not wrapped around the nerve for purposes of securing other wraps in position, for example, similar to the example of FIG. 10.

The implantable medical apparatuses of the disclosure may also achieve electrical capture relative to the target tissue where there was no electrical capture previously. In some of these examples, the location at which the plurality of electrodes or electrode arrangement are introduced (e.g. implanted) may comprise nerve branches related to, extending from, etc. the nerve branch on which the previously implanted electrode arrangement is located. In some of these examples, the additionally placed electrodes may be added on the same side of the body as a previously placed first electrode arrangement (e.g. unilateral deployment). However, in some of these examples, the additionally placed second electrode arrangement may be added on a different side of the body relative to the previously implanted electrode arrangement, thereby achieving bilateral deployment.

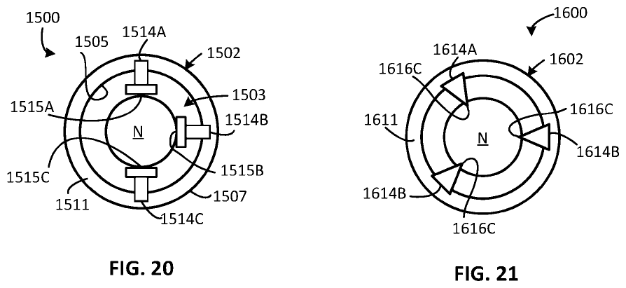

FIG. 20 illustrates an example implantable medical apparatus 1500 which may achieve electrical capture relative to the target tissue where there was no electrical capture previously. It is to be understood that other aspects of the implantable medical apparatus 1500 of FIG. 20 can be similar or identical to that of FIGS. 1A-19 and that other features of other embodiments disclosed herein may also be incorporated into the embodiment of FIG. 20. In one example, the implantable medical apparatus 1500 includes a wrap 1502 comprising a nonconductive carrier body 1511 supporting a plurality of electrodes 1514A, 1514B, 1514C (e.g., stimulation electrodes) extending radially inwardly with respect to a lumen 1503 of the wrap 1502. In various examples, one or more electrodes 1514A-1514C can be positioned at the same longitudinal position with respect to the carrier body 1511. In other examples, one or more electrodes 1514A-1514C can be positioned at varying longitudinal positions with respect to the carrier body 1511. When including one or more electrodes (e.g., 1514A-1514C), a wrap may comprise a cuff in some examples. In various examples, the wrap 1502 comprises a nonconductive body 1507 either forming an enclosed lumen 1503 either via a continuous circumference of material or via an overlapping portion or the like (see also, overlapping portion 912 and wrap 902 of FIG. 7, for example). In various examples, the plurality of electrodes 1514A-1514C are radially spaced from each other (evenly spaced or unevenly spaced) within the lumen 1503, which may allow for different stimulation vectors and/or selectively activating different bundles within target nerve N. The wrap 1502 can otherwise include any features or elements of previously disclosed wraps (e.g., wrap 902) and can be used in a similar manner to previously disclosed wraps. In one example, one or more of the electrodes 1514A-1514C may have a T-shape with a flat contact surface 1515A, 1515B, 1515C. In various examples, each of the plurality of electrodes 1514A-1514C is biased away from an inner surface 1505 of the wrap 1502 in a direction toward a central axis of the lumen 1503. In various examples, the lumen 1503 is configured to have substantially the same diameter as the target nerve N (e.g., a diameter within 0.10-5 mm of a diameter of the target nerve).

As shown in FIG. 21, in another example, an alternate implantable medical apparatus 1600 can include a wrap 1602 having a nonconductive carrier body 1611 supporting a plurality of electrodes 1614A, 1614B, 1614C. In some examples, at least one electrode 1614A-1614C comprises a pyramid shape, such as comprising a triangular cross-section, and/or are otherwise tapered and/or include a pointed contact surface/tip 1515A, 1515B, 1515C for contacting/engaging the target nerve N. Three electrodes 1514A-1514C are illustrated, however, more or fewer electrodes can be utilized. The example of FIG. 21 may, in some variations, otherwise be identical to that of FIG. 20. In either of the examples of FIG. 20-21 or in some other examples of the disclosure, the plurality of electrodes can be evenly or unevenly spaced about the inner circumference of the carrier body. Use of the implantable medical apparatuses of FIGS. 20-21 can include any of the steps disclosed herein with respect to other embodiments utilizing one or more wraps and including engaging the plurality of electrodes of the wrap with target tissue. In various examples, the plurality of electrodes are sided, shaped, orientated and/or biased so that when the wrap becomes removalby secured relative to a nerve, at least a contact surface of each of the plurality of electrodes pressingly engages the nerve to ensure robust contact with the nerve to increase the likelihood of electrically engaging the nerve.

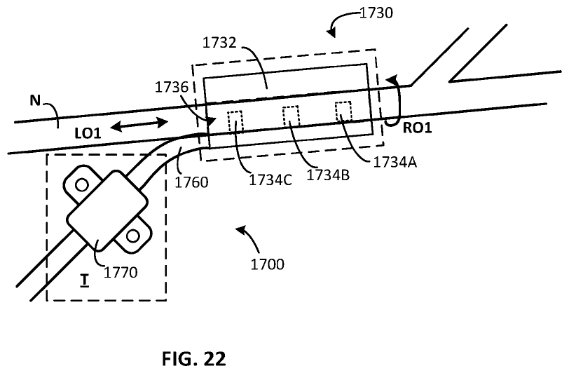

FIG. 22 illustrates implantable medical apparatus 1700 according to one example of the disclosure. In this example, the implantable medical apparatus 1700 includes a flexible lead body 1760 connected to an electrode arrangement 1730 having a nonconductive carrier body 1732 maintaining one or more electrodes 1734A, 1734B, 1734C to have a contact surface of each electrode 1734A-1734C exposed at a lumen 1736 of the carrier body 1732 (see also FIGS. 20-21, for example, as well as FIG. 3A and similar electrode arrangement 630). In one example, the structure of the electrode arrangement 1730 may be described as a cuff electrode although other electrode arrangement structures of the disclosure can be substituted for the electrode arrangement of FIG. 22. In any case, the electrode arrangement 1730 is configured to be secured to nerve N in an electrically engaging relation for sensing and/or stimulation.

In one example, the lead body 1760 includes one or more anchors 1770 for securing the implantable medical apparatus 1700 to tissue T that may be adjacent the target nerve N. Each anchor 1770 can be secured to the tissue T with sutures or other surgical fastening mechanisms. It is to be understood that other aspects of the implantable medical apparatus 1700 of FIG. 22 can be similar or identical to that of FIGS. 1A-21 and that other features of other embodiments disclosed herein may also be incorporated into the embodiment of FIG. 22.

Methods of using the implantable medical apparatus 1700 can include forming an incision and positioning the electrode arrangement 1730 in position to achieve an electrically engaging relation to the nerve N. Some example methods may include wrapping the carrier body 1732 around the nerve N. The lead body 1760 can be bent, curved or reshaped to achieve the desired positioning of the electrode arrangement 1730 with respect to the nerve N. At a time appropriate to chronically maintain a desired rotational orientation (arrow RO1) and/or a desired longitudinal positioning (LP1), via anchor(s) 1770 the implantable medical apparatus 1700 can be anchored to tissue T adjacent the target nerve N. Among other aspects, this example suture/lead arrangement may enhance maintaining a rotational/longitudinal position of the electrodes 1734A, 1734B, 1734C relative to the targeted fibers within the nerve N and/or to maintain a rotational/longitudinal position of the carrier body 1732 in the midst of various surrounding anatomical structures.

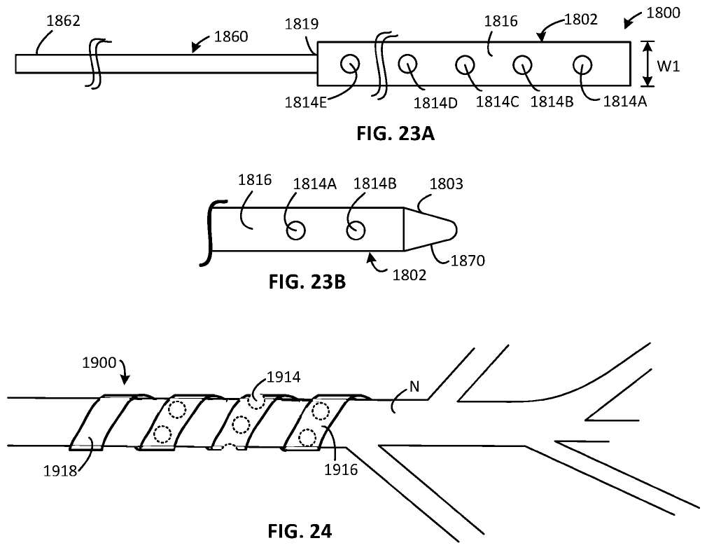

FIGS. 23A-23B schematically illustrate example implantable medical apparatus 1800 that may achieve electrical capture relative to the target tissue where there was no electrical capture previously. In this example, the implantable medical apparatus 1800 includes an electrode arrangement 1802 including an elongate flexible carrier body 1816 supporting a plurality of electrodes 1814A, 1814B, 1814C, 1814D, 1814E (e.g., stimulation electrodes). In alternate examples, the electrode arrangement 1802 may include more or fewer electrodes. The plurality of electrodes 1814-A-1814E can be arranged in series, either evenly spaced or unevenly spaced, in various examples. Additionally, the electrodes 1814A-1814E can be aligned along a midpoint of a maximum width W1 of the carrier body 1816 or at least one electrode can be offset with respect to the midpoint. The carrier body 1816 may be formed of a nonconductive material and connected to a lead body 1860. In various examples, the carrier body 1816 includes conductors for each electrodes 1814A-1814E that may extend to and through (e.g. along a length of) the lead body 1860. In one example, the lead body 1860 includes a proximal portion 1862 connected to a transition portion 1819, which interconnects the proximal portion 1862 to the carrier body 1816. In one example, the most proximal electrode 1814E is spaced at least one electrode diameter length away from a transition portion 1819. Both the proximal portion 1862 and/or the transition portion 1819 may have a greater rigidity as compared to the carrier body 1816 according to various examples of the disclosure. Additionally, the transition portion 1819 and proximal portion 1862 may have differing cross-sectional shapes and/or sizes as compared to the carrier body 1816.

In some examples, the carrier body 1816 and/or the lead body 1860 are made of a resilient material so that they can be wrapped around a nerve. In some examples, the carrier body 1816 may include a flexible but non-resilient material such that once wrapped about a nerve, it maintains the curvatures formed in the carrier body 1816 so that the carrier body 1816 does not become unwrapped from the nerve. In some examples, the lead body 1860 can be similarly configured for wrapping either around the nerve or other bodily structures.

As shown in FIG. 23B, in some examples a distal end 1803 of the electrode arrangement 1802 comprises an atraumatic tip 1870, which can be domed or tapered in some examples. It is to be understood that other aspects of the implantable medical apparatus 1800 of FIGS. 23A-23B can be similar or identical to that of FIGS. 1A-22 and that other features of other embodiments disclosed herein may also be incorporated into the embodiments of FIGS. 23A-23B.

Example methods of using the implantable medical apparatus 1800 of FIGS. 23A-23B can include forming an incision in a body (see also, FIG. 2A and related disclosure) and positioning the electrode arrangement 1802 adjacent target tissue (e.g., along a nerve) so that the plurality of electrodes 1814A-1814E are in electrical communication with the target tissue for sensing and/or stimulation.

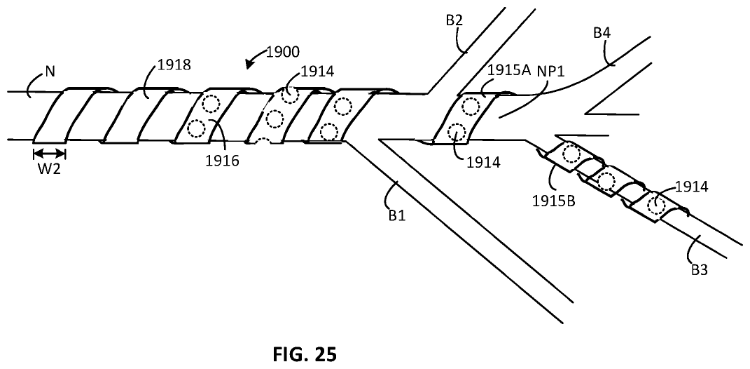

FIGS. 24-25 illustrate implantable medical apparatus 1900 according to an example of the disclosure. In this example, the implantable medical apparatus 1900 includes an electrode arrangement 1902 with a plurality of electrodes 1914 maintained within a flexible carrier body 1916. In FIG. 24, the plurality of electrodes 1914 are generally referenced and are positioned on an underside surface of the carrier body 1916. The plurality of electrodes 1914 are positioned within the carrier body 1916 in such a way that a contact surface of each electrode is exposed. The carrier body 1916 can be akin to a ribbon in its shape and flexible nature such that the carrier body 1916 is suitable for wrapping around a nerve N in a generally helical fashion, as shown, so that the contact surface of the electrodes 1914 are in electrical communication with the nerve. For example, the electrode arrangement 1902 can be repeatedly wound over a distance that is not necessarily linear and not necessarily having a uniform curves). In examples such as that of FIG. 25, the carrier body 1916 may be wrapped around multiple nerve branches (e.g., one or more of B1, B2, B3, B4) so that multiple nerve branches can be accessed with a single electrode arrangement/apparatus. Various examples of the disclosure, including that of FIG. 25 allow for dynamic maneuvering of the implantable medical apparatus to accommodate varying types of bodily structures/target tissues, provide custom fitting to ensure electrical engagement and increase the ease in which the implantable medical apparatus can be operatively implanted. Such examples can allow access to a wide variety of nerve portions (e.g., electrodes 1914 at portion 1915A can engage nerve portion NP1), that may not otherwise be accessible with other electrode arrangements having less versatile geometry. For example, a distal nerve branch B3 can be accessed with portion 1915B of the implantable medical apparatus 1900 while nerve portion NP1 and portions to the left of branch B1 are also engaged by the same implantable medical apparatus 1900.

The plurality of electrodes 1914 can be provided to achieve sensing and/or stimulation of the target tissue. In various examples, the plurality of electrodes includes 5 or more electrodes or 6 or more electrodes. Other examples may include more or fewer electrodes. In some examples, a distance between adjacent electrodes is minimized to achieve a minimum spacing which will allow the flexible carrier body 1916 to remain flexible for wrapping around the target nerve N given the electrodes 1914 and associated wiring present within the carrier body 1916. The plurality of electrodes can optionally be configured as shown in with respect to the examples of FIGS. 23A-23B. In some examples, the flexible carrier body 1916 may include a proximal lead portion 1918 that is free of electrodes but may otherwise be identically configured to carrier body 1916 or differently configured, according to various examples of the disclosure. In some examples, the proximal portion 1918 may be free of electrodes but may include conductive elements for the respective electrodes 1914.

With this in mind, in some examples, one or more electrodes 1914 may comprise a printed electrode arrangement, which has at least one contact electrode on each side of a generally flat carrier body. In some examples, having contact electrodes on both (opposite) sides of the carrier body 1916 may expand the number, type, orientation, location, etc. of potential stimulation vectors (among the multiple spaced apart contact electrodes) which may be identified and used to apply stimulation.

In some examples, a printed electrode arrangement may comprise a relatively thin, low profile insulating carrier body 1916 formed in the manner of a printed circuit board construction including conductive traces to act as conductors to and between respective contact electrodes 1914. In some examples, the printed electrode arrangement may be flexible which may sometimes be referred to as flexible printed circuit-type elements. In some such examples, the printed electrode arrangement omit other more complex forms of circuitry, such as pulse generating circuitry (e.g. stimulation signal forming circuitry), wireless communication circuitry, and the like. Among other aspects, the low profile (e.g. low thickness or diameter) of the printed electrode arrangement may facilitate introduction and advancement of the printed electrode arrangement in and among target tissues.

In some examples, at least some lead segments (e.g. lead body) also may be formed as printed circuit-type elements with each lead segment comprising an insulating substrate on which is printed conductive traces to carry a signal from or to a printed electrode arrangement (e.g. including electrodes 1914 on carrier body 1916) relative to a pulse generator or other stimulation circuitry or sensing circuitry. The printed conductive traces are covered with an insulating jacket or coating, which may cover just the conductive traces or the entire assembly of the substrate and conductive traces. In some examples, the insulating jacket also may comprise part of the “printing” of the lead segments.

In some such examples, printed lead segments and printed electrode arrangement may be formed as a single, unitary (e.g. monolithic) construction.

In some such examples, the printed lead segments may have a cross-sectional profile, such as a shape (e.g. rectangular, circular, elliptical, etc.) and/or dimensions (e.g. width, height, diameter, greatest cross-sectional dimension, etc.) which are generally the same as a cross-sectional profile (e.g. shape and/or dimensions) of the printed electrode arrangement. In some examples, this general matching of the cross-sectional profiles of the lead segments and the printed electrode arrangement may be implemented even for example implementations in which the lead segments and/or the electrode arrangement (e.g. including contact electrodes) are not printed circuit-type elements. In some examples, whether printed or not, the general matching of the cross-sectional profile of the respective lead segments with the electrode arrangement (e.g. including contact electrodes) may facilitate certain types of anchor structures, such as but not limited to at least some examples anchor structures of the present disclosure.

However, in some examples, the cross-sectional profile (e.g. shape and/or dimensions) of the lead segments may be different from the cross-sectional profile of the printed electrode arrangement. This relationship may be implemented in examples in which one or both of the lead segments and the electrode arrangement are printed circuit-type elements or may be implemented in examples in which none of the lead segments or electrode arrangement are printed circuit-type elements.

In various examples, the implantable medical apparatus 1900 of FIGS. 24-25 include a relatively high number of electrodes 1914 (e.g., stimulation electrodes) over a relatively long length of the carrier body 1916. The carrier body 1916 may have a uniform width W2 in some examples. In some examples, the width W2 of the carrier body 1916 is no more than 50% greater than a diameter (or greatest cross-sectional dimension) of each respective electrode 1914. In some examples the non-conductive material positioned between electrodes (i.e. carrier body 1916) is greater than 50% of the diameter of the electrodes 1914 (or greatest cross-sectional diameter). In some examples, the carrier body 1916 can define a pre-formed helical coil in a natural arrangement that could be a predefined percentage (e.g., 25%-500%) of the diameter of the target nerve N.

It is to be understood that other aspects of the implantable medical apparatus 1900 of FIGS. 24-45 can be similar or identical to that of FIGS. 1A-23B and that other features of other embodiments disclosed herein may also be incorporated into the embodiments of FIGS. 24-25.

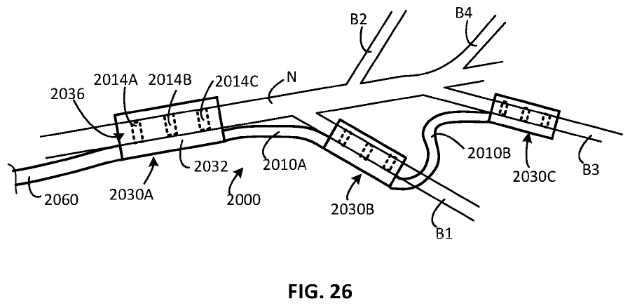

FIG. 26 illustrates implantable medical apparatus 2000 according to an example of the disclosure. In this example, the implantable medical apparatus 2000 includes a plurality of electrode arrangements 2030A, 2030B, 2030C interconnected in series with flexible spacers 2010A, 2010B. A lead body 2060 extends from the most proximal electrode arrangement 2030A and can house conductors for each of the electrode arrangements 2030A-2030C. In some such examples, the spacers 2010A, 2010B may comprise and/or sometimes be referred to as portions of lead body 2060, i.e. lead portions 2010A, 2010B. In some examples, each electrode arrangement 2030A-2030C may be a cuff-style electrode arrangement similar to that of FIG. 3A. In various examples, each electrode arrangement 2030A-2030C may include one or more electrodes 2014A, 2014B, 2013C (e.g., stimulation electrodes) within a nonconductive carrier body 2032 according to an example of the disclosure. The carrier body 2032 can define a lumen 2036 in which the nerve N can be positioned to achieve an electrically engaged relation between the respective electrode arrangement 2032A and the nerve N. Only one electrode arrangement 2030A is fully referenced for ease of illustration but it will be understood that other electrode arrangements (2030B, 2030C, etc.) may be identically configured in various examples but this is not required. In some examples, the spacer(s) 2010A, 2010B interconnecting the electrode arrangements 2030A-2030C can include an electrical conductor corresponding to a respective one of the electrodes 2014A-2014C in each respective carrier body 2032. Each spacer 2010A, 2010B can be akin to the flexible body 1916 of FIG. 25 according to one example of the disclosure. Although the implantable medical apparatus 2000 is shown as having three electrode arrangements 2030A-2030C, more or fewer electrode arrangements can be provided. It is to be understood that other aspects of the implantable medical apparatus 2000 of FIG. 26 can be similar or identical to that of FIGS. 1A-25 and that other features of other embodiments disclosed herein may also be incorporated into the embodiment of FIG. 26.

One method of using the implantable medical apparatus 2000 of FIG. 26 can include positioning the electrode arrangements 2030A, 2030B, 2030C of the implantable medical device 2000 around a nerve N in series, one by one. In one example, at least two of the electrode arrangements 2030A-2030C of the implantable medical device 2000 are positioned on different branches B1, B2, B3 of the nerve N. In one example, at least three of the electrode arrangements 2030A-2030C of the implantable medical device are positioned on different branches B1-B3 of the nerve N and connected to a pulse generator via lead body 2060.

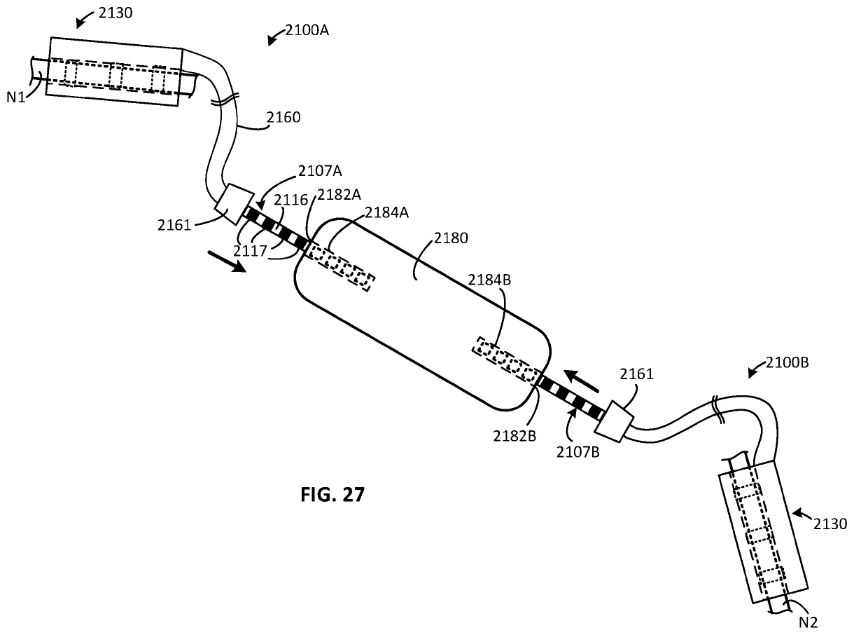

FIG. 27 schematically illustrates an alternate first and second implantable medical apparatus 2100A, 2100B for use with a pulse generator 2180 (e.g. implantable pulse generator (IPG)) according to an example of the disclosure. The implantable medical apparatus 2100A may, in some examples, be identical in configuration to implantable medical apparatus 2100B. In the present example, implantable medical apparatuses 2100A and 2100B are identical and, therefore, only one implantable medical apparatus 2100A is fully labeled for ease of illustration. In one example, the pulse generator 2180 is implemented as a microstimulator. In one example, the implantable medical apparatus 2100A can include a proximal connector electrode arrangement 2107A interconnected to a second electrode arrangement 2130 via a lead body extension 2160. The implantable medical apparatus 2100A can alternatively be any of the types disclosed herein. The proximal connector electrode arrangement 2107A can be an axial electrode having a plurality of electrodes 2117 (generally referenced) supported by a carrier body 2116 or may have any other configuration disclosed herein suitable for connection to the pulse generator 2180 at a first location 2182A. Further, the proximal connector electrode arrangement 2107A and the pulse generator 2180 are collectively configured such that the connector electrode arrangement 2107A can be releasably engaged with the pulse generator 2180 for later removal or disconnection, if desired. Should the electrical capture of a first target nerve N1 by the second electrode arrangement 2130 be insufficient or another nerve capture is desired, the pulse generator 2180 is configured to releasably receive a proximal connector electrode arrangement 2107B of the second implantable medical apparatus 2100B at a second location 2182B in a housing of the pulse generator. Therefore, the pulse generator 2180 is configured to receive two implantable medical apparatuses 2100A, 2100B simultaneously so that multiple discrete and separated nerve/tissue targets (e.g., N1, N2) can be stimulated/sensed by a single pulse generator 2180. In some examples, the pulse generator 2180 may be configured to receive more than two implantable medical apparatuses 2100A, 2100B simultaneously. The second electrode arrangement 2130 is a cuff configuration, similar to electrode arrangement 630 of FIG. 3A, in the illustrated example but can take any other configuration disclosed herein for attachment to a nerve. In this example, the pulse generator 2180 may be implanted at a location in a patient's body, such as a head or neck or anywhere identified in FIG. 1B.

In one example, the first location 2182A and the second location 2182B are on opposite ends of the pulse generator 2180. In some examples, the pulse generator 2180 includes a recess/port 2184A, 2184B at the first and second locations 2182A, 2182B in which one respective first electrode arrangement (e.g., 2107A) can at least partially be inserted. In one example, at least one recess/port can 2184A, 2184B include a cover, plug or the like to prevent fluid ingress until one electrode arrangement is inserted within the port/recess 2184A, 2184B. The plug or alternate cover can be repositioned for access to the respective port, as desired.

Interconnecting the lead body 2160 and the proximal connector electrode arrangement 2107A may be a transition portion 2161. The transition portion 2161 may have a greatest outer dimension that is larger than a greatest outer dimension of one or more of the lead body 2160 and the proximal connector electrode arrangement 2107A. In some examples, the transition portion 2161 may function to seal the port 2184A when the first electrode arrangement 2107A is operatively engaged with the pulse generator 2180. Therefore, in various examples, the transition portion 2161 may be made of a liquid impermeable, elastic/resilient material.

It is to be understood that other aspects of the first and/or second implantable medical apparatuses 2100A, 2100B or pulse generator 2180 of FIG. 27 can include similar or identical features to that of FIGS. 1A-26 and that other features of other embodiments disclosed herein may also be incorporated into the embodiment of FIG. 27.

In one example method of the disclosure, the pulse generator 2180 is surgically implanted within a patient's body. The first implantable medical apparatus 2100A is positioned with the patient's body so that the second electrode arrangement 2130 is believed to be sufficiently positioned to electrically engage and communicate with a target nerve N1 and the proximal connector electrode arrangement 2107A is connected to the pulse generator 2180. The order of these two steps can be reversed. In one example, during the same or during a second surgical procedure (in which a second incision is formed at a later date), the second implantable medical apparatus 2100B is inserted through the incision and positioned within the patient's body so that a second electrode arrangement 2104B is believed to be sufficiently positioned to electrically capture the second target nerve N2 and the second electrode arrangement is connected to the pulse generator at a location different from the first implantable medical apparatus 2100A. In some examples, N1 and N2 may be different portions of the same tissue. In various examples in which more than two ports 2184A, 2184B are provided in the pulse generator 2180, methods may include connecting additional implantable medical apparatuses to such additional ports of the pulse generator, as desired, which may or may not include removal of a plug prior to connection. Some example methods may additionally include disconnecting one or more previously implantable medical apparatuses from the respective nerve and removing the implantable medical apparatus from the patient body for either abandonment of that particular therapeutic approach or for replacement with an alternate implantable medical apparatus. Some example methods may further include relocating one or more previously implanted medical apparatuses (2100A, 2100B) from one port (2184A, 2184B) to another port (2184A, 2184B) to accommodate introduction of a new lead/electrode arrangement.

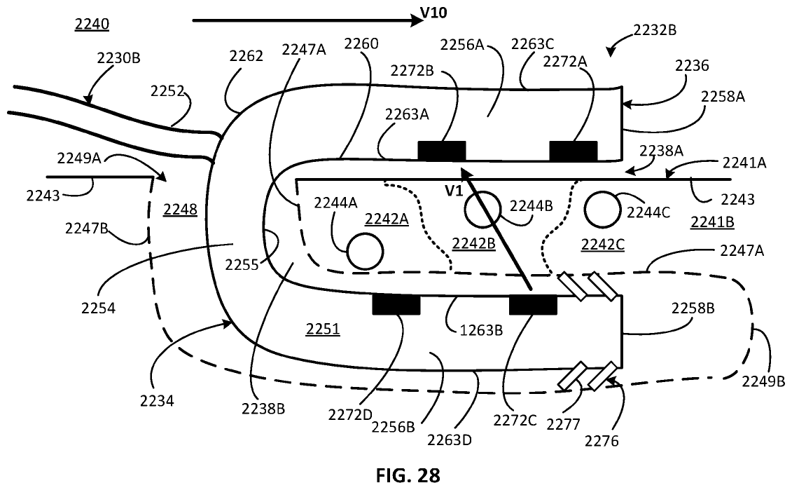

FIG. 28 illustrates part of an alternate second electrode arrangement 2200 that can be incorporated into any of the implantable medical apparatuses of the disclosure. As just one example, in the example arrangement of FIGS. 3A, 3C, the second electrode arrangement 2200 may be used instead of the second electrode arrangement 630 or in the example arrangement of FIG. 5A, the second electrode arrangement 2200 may be used instead of second electrode arrangement 830. It is to be understood that aspects of FIG. 34B can be similar or identical to that of FIGS. 1A-27 and that other features of other embodiments disclosed herein may also be incorporated into the embodiments of FIG. 28.

FIG. 28 schematically represents an electrode arrangement 2232B in stimulating relation to target tissues (e.g. target nerve portions, target muscle portions, and/or target neuromuscular junctions). The electrode arrangement 2232B is connected to a lead body extension 2230B, which can be any lead body extension in examples of the disclosure, and which may have a dock (e.g. 638, 738, 838 in FIGS. 3A-5) on the opposing end of the lead body extension 2230B.

In some examples stimulation element 2232B may comprise a distal portion 2252 of distal lead segment 2230B with the stimulation element 2232B acting to mechanically and electrically support contact electrodes to engage target tissues. In some examples, the tissue-engaging stimulation element 2232B comprises a U-shaped body 2251 including a base 2254 (at proximal end 2234 of stimulation element 2232B) and a pair of arms 2256A, 2256B which extend from base 2254 in a spaced apart relationship in some examples. In some examples, the spaced apart, arms 2256A, 2256B may be in a generally parallel relationship as shown in FIG. 2B before, during, and/or upon chronic implantation, while in some examples, the spaced apart arms 2256A, 2256B may form a non-parallel angle before, during, and/or upon chronic implantation.

In some examples, the U-shaped body 2251 comprises an outer surface 2262 and an inner surface 2260, which acts as a contact surface against or with target tissue portions. An end 2258A, 2258B of respective arms 2256A, 2256B of the tissue-engaging stimulation element 2232B corresponds to the distal end 2236 of the stimulation element 2232B. Moreover, a distal opening 2238A (i.e. distal open end) of the U-shaped body 2251 is defined by the open space between the ends 2258A, 2258B of the arms 2256A, 2256B at the distal end 2236. In some examples, a channel 2238B is defined by the space between the arms 2256A, 2256B.

In some instances, the electrode arrangement 2232B may comprise individually addressable contact electrodes (e.g. 2272A-2272D) at least to the extent that in some examples, the contact electrodes may be in stimulating relation via a target tissue, such as being in direct contact with a target nerve portion(s), target muscle portion(s), combination of a target nerve portion(s) and target muscle portion(s), neuromuscular junction of the target nerve portion and target muscle portion, and/or combinations thereof. In some such examples, the ends of the nerve portion(s) forming the neuromuscular junction(s) may sometimes be referred to as nerve ending(s).

As shown in FIG. 28, the stimulation element 2232B may comprise an anchor structure 2276 on the body 2251 to secure the stimulation element 2232B relative to tissues 2241A, 2241B, which may comprise non-nerve tissue in some examples. In some examples, the anchor structure 2276 may comprise tines 2277 or other elements which engage surrounding non-nerve tissues to anchor the respective arm (e.g. 2256B) relative to the non-nerve tissue, and therefore relative to target tissue(s) 2241A, which comprises muscle portions 2242A, 2242B, 2242C and nerve portions 2244A, 2244B, 2244C (shown in cross-section).

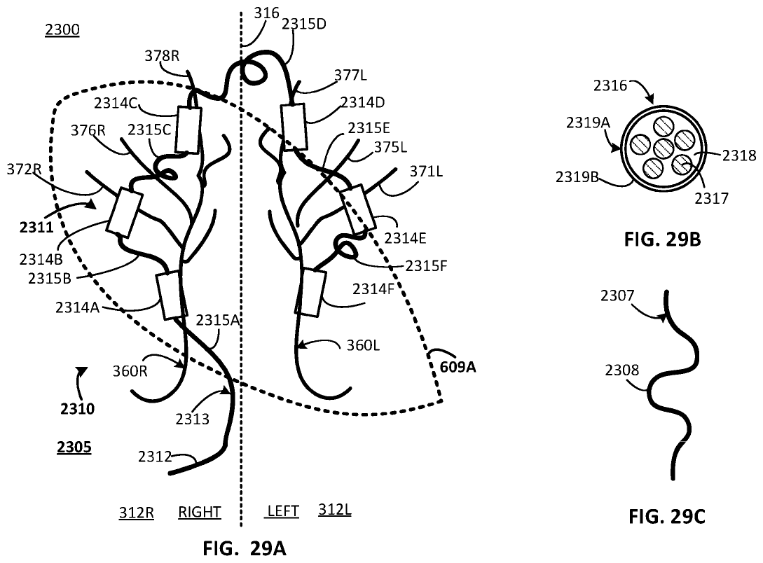

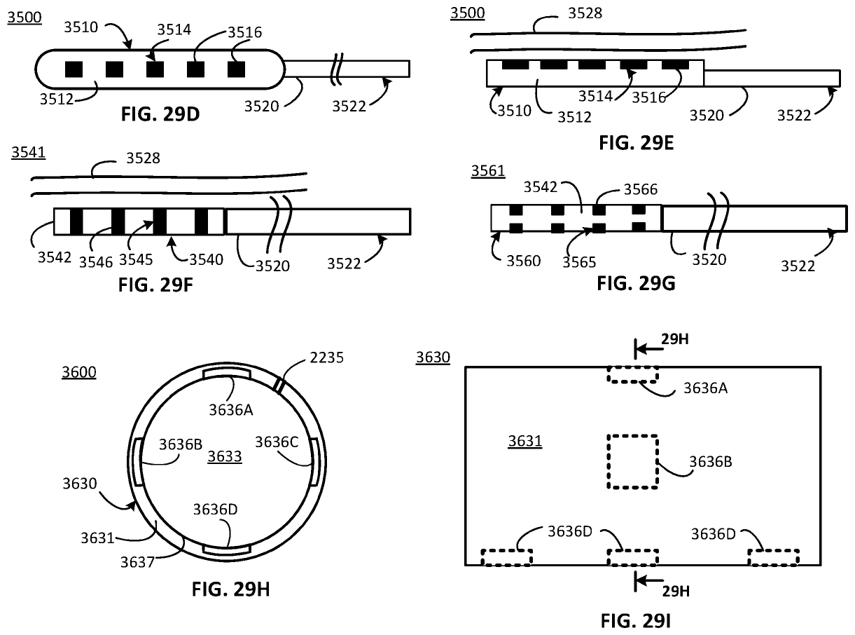

FIGS. 29A-29C illustrate a diagram 2300 schematically representing an example method and/or example device 2305 for treating sleep disordered breathing via a stimulation portion 2311 comprising a connected array of stimulation elements 2314A-2314F. In some examples, the stimulation portion 2311 can be incorporated into any of the implantable medical apparatuses of the disclosure. As just one example, in the example arrangement of FIGS. 3A, 3C, the stimulation portion 2311 (FIGS. 29A-29C) may be used instead of the second electrode arrangement 630 or in the example arrangement of FIG. 5A, the stimulation portion 2311 (FIGS. 29A-29C) may be used instead of second electrode arrangement 830. It is to be understood that aspects of FIGS. 29A-29C may be similar or identical to that of FIGS. 1A-28 and that other features of other embodiments disclosed herein may also be incorporated into the embodiment of FIGS. 29A-29C. As shown in FIG. 29A, one example method and/or example device 2305 comprises a stimulation lead 2310 being implanted via a midline implant-access incision 609A with lead 2310 including a stimulation portion 2311 including a single strand of stimulation elements 2314A-2314F.

Among other aspects, the example device 2305 may provide for a highly customizable implantation of multiple stimulation elements among several different target tissues (e.g. target nerve portions, target muscle portions, combinations of nerve portions and muscle portions, neuromuscular junctions of target nerve portion(s) and target muscle portion(s), and/or combinations thereof) in order to apply stimulation selectively and independently to different target tissues and/or in order to apply stimulation via various combinations of the implanted stimulation elements to achieve desired stimulation vectors. The stimulation may be applied among the different stimulation elements in different manners, such as (but not limited to) sequentially, simultaneously, alternating, bilaterally, unilaterally, and/or via other patterns.

In some examples, the example method and/or example device 2305 comprises the stimulation portion 2311 of a connected array of spaced apart stimulation elements 2314A-2314F with each stimulation element 2314A-2314F being independently positionable into stimulating relation to different portions of upper airway patency-related tissue. As shown in FIG. 29A, the stimulation portion 2311 comprising the connected array may be supported by or on a distal portion 2313 of stimulation lead/lead body extension 2310. In some examples, the distal portion 2313 may sometimes be referred to as distal lead portion 2313. In some examples, the stimulation portion 2311 may comprise a greater number or fewer number of stimulation elements than shown in FIG. 29A as 2314A-2314F.

While it will be understood that the distal lead portion 2313 provides a continuous length including electrical conductors (covered by an insulative jacket), when viewed from a proximal to distal orientation, the distal lead portion 2313 may referred to as including various lead segments (e.g. 2315A, 2315B, etc.) which are exposed (e.g. present) prior to, between, or after a stimulation element (e.g. 2314A, 2314B, etc.). For instance, in some examples the distal lead portion 2313 may comprise one exposed lead segment 2315A being located just proximal to a stimulation element 2314A, an exposed lead segment 2315B being located between stimulation elements 2314A, 2314B, and exposed lead segment 2315C located between stimulation elements 2314B, 2314C, and so on. It will be further understood that the distal lead portion 2313 comprises an array of electrically independent conductors extending therethrough with each independent conductor in electrical communication and connection with a corresponding stimulation element (e.g. 2314A, 2314B, etc.) such that the respective stimulation elements 2314A-2314F are independently controllable for applying a stimulation signal to different target tissue portions (e.g. nerve, muscle, or nerve-muscle combination). One example implementation is shown later in FIG. 29B.

As further shown in FIG. 29A, in some examples, the distal lead portion 2313 is implanted such that at least some of the respective stimulation elements 2314A-2314F are chronically positioned in stimulating relation to target tissues, with several stimulation elements (e.g. 2314A, 2314B, 2314C) are fixed in stimulating relation to target tissues on one side (e.g. right side 312R) of the patient's body while several stimulation elements (e.g. 2314D, 2314E, 2314F) are fixed in stimulating relation to target tissues on another side (e.g. left side 312L) of the patient's body. The number of stimulation elements on one side (e.g. right side 312R) of the patient's body may be the same as, or different from, the number of stimulation elements on the opposite, other side (e.g. left side) of the patient's body.

Moreover, in some examples, the stimulation elements implanted on one side (e.g. right side) of the patient's body may be positioned at generally the same target tissue locations as the position of the respective implanted stimulation elements located on the other side (e.g. left side) of the patient's body. However, in some examples, the stimulation elements implanted on one side (e.g. right side) of the patient's body may be positioned in at least some target tissue locations which are different from at least some of the target tissue locations at which implanted stimulation elements on the other side (e.g. left side) of the patient's body are positioned.

In some examples, each stimulation element 2314A-2314F may comprise a carrier supporting a single contact electrode. The carrier may comprise an insulating material and is configured to permit electrical connection between the contact electrode and the electrical conductor(s) extending through the distal lead portion 2313 on which the stimulation element (e.g. 2314A, 2314B, etc.) is mounted. However, in some examples, each stimulation element may comprise multiple contact electrodes spaced apart from each other on the carrier. As further described later in association with at least FIGS. 29D-291, the carrier and contact electrodes of any one of (or all) the stimulation elements 2314A-2314F may be implemented in a variety of configurations including carriers supporting ring electrodes, split-ring electrodes, cuff electrodes, etc. as well as being configured as in the example of FIG. 28, or the like. In some examples, at least one of the stimulation elements 2314A-2314F may comprise an electrode arrangement such as the electrode arrangements 630, 830 in FIGS. 3A and 5, respectively.

As shown in FIG. 29A, in some examples at least some of the stimulation elements 2314A, 2314B, etc. are positioned to directly overlap with a nerve portion, such as stimulation element 2314A directly overlapping with nerve (portion) 360R, stimulation element 2314B directly overlapping with nerve portion 372R, stimulation element 2314C directly overlapping with nerve portion 378R, stimulation element 2314D overlapping with nerve portion 377L, stimulation element 2314E overlapping with nerve portion 371L, stimulation element 2314F overlapping with nerve (portion) 360L.

However, in some examples, some stimulation elements may not necessarily directly overlap with a nearby nerve portion, such as one of the stimulation elements being in close proximity to (but not directly overlapping) a nerve portion. This arrangement may occur because of the particular patient anatomy, size and/or shape of the stimulation elements, etc. Nevertheless, to the extent that the particular non-overlapping stimulation element may be close enough to the target nerve or target neuromuscular junction, the stimulation element may still be in stimulating relation to at least the target nerve portion, target muscle portion, and/or target neuromuscular junction. Moreover, even if the stimulation element is not in stimulating relation to a nearby target nerve and/or neuromuscular junction, the stimulation element may be close enough to a target muscle portion to be in stimulating relation to the target muscle portion (e.g. portion of the genioglossus muscle) suitable to positively affect upper airway patency.

Moreover, in some examples, at least some target tissue (e.g. nerve portion, muscle portion, combinations of nerve portions and muscle portions, neuromuscular junctions, and/or combinations thereof) may be stimulated via stimulation vectors applied across, applied through, etc. target tissue which is located between or near spaced apart stimulation elements (e.g. 2314A-2314F) of the stimulation lead 2310.

In some examples, an example method of using a device (e.g. 2305) comprises implanting the distal lead portion 2313 via a midline implant-access incision 609A using direct visualization techniques (within minimal or no tunneling) enabled by a size and/or shape of the midline implant-access incision. However, it will be understood that in some instances, tools may be used to form implant paths, partial tunnels or pockets to enable positioning at least some stimulation elements (e.g. 2314A, etc.) relative to pertinent target tissues. It also will be understood that implant-access incision 609A may be implemented at locations other than a sagittal midline location.

In some examples, the lead segments (e.g. 2315A, 2315, etc.) between adjacent stimulation elements (e.g. 2314A, 2314B, etc.) may comprise a flexibility greater than a flexibility of lead body/lead body extension (e.g. 2312) proximal to the entire group of stimulation elements. In some such examples, this arrangement may facilitate maneuvering, positioning, and/or orienting the respective stimulation elements within and among target tissues during implantation.

In some examples, the lead segments (e.g. 2315A, 2315B, etc.) comprise a greatest cross-sectional dimension which is less than a greatest cross-sectional dimension of the stimulation elements 2314A-2314F. The lead segments 2315A, 2315B, etc. may have a cross-sectional shape which is the same as, or different from, a cross-sectional shape of the stimulation elements 2314A-2314F. In some examples, the lead segments (e.g. 2315A, 2315B, etc.) comprise a greatest cross-sectional dimension which is substantially less than a greatest cross-sectional dimension of the stimulation elements 2314A-2314F. In some such examples, the substantially less difference comprises a difference of at least 25 percent or at least 50 percent. In some examples, this arrangement which may enhance the flexibility and maneuverability of the lead segments (e.g. 2315A, 2315B, etc.) relative to the stimulation elements 2314A-2314F, which in turn may enhance the maneuverability of stimulation elements 2314A-2314F relative to the target tissues and/or of the stimulation elements 2314A-2314F relative to each other.

In some examples, the respective lead segments may comprise lengths (e.g. spacing) between adjacent stimulation elements (e.g. 2314A, 2314B, etc.) and/or any anchor structures.

FIG. 29B is a sectional view schematically representing an example lead segment 2316, which may comprise an example implementation of any one or all of the lead segments 2315A-2315F. As shown in FIG. 29B, in some examples, the example, lead segment 2316 may comprise a flexible electrically insulative conduit (e.g. hollow tubular structure) including a side wall 2319A defining an outer surface 2319B. In some examples, a plurality of electrical conductors 2317 extend through and within an interior 2318 of the conduit to establish electrical connection between a pulse generator (or a more proximal lead portion) and a respective contact electrode of one of the respective stimulation elements (e.g. 2314A, 2314B, etc.). It will be understood that, in some examples, a single conductor 2317 (or single conductor group) supports and is electrically connected to a single stimulation element (e.g. 2314A) to enable independently addressable control of individual electrodes of the respective stimulation elements in applying stimulation to the respective target tissues. The interior 2318 may comprise insulative material to electrically insulate the respective conductors 2317 from each other.

In some examples, upon electrical connection of a respective one of the conductors 2317 to a respective one of the more proximal stimulation elements, such as stimulation element 2314A, the respective conductor 2317 may terminate at the location of such electrical connection. In this arrangement, the particular conductor 2317 does not extend distally from the respective stimulation element 2314A while the other respective conductors 2317 extend distally further within and through the conduit (as defined by side wall 2319A) to reach their own respective stimulation element (e.g. 2314B, 2314C, etc.). In a manner similar to the stimulation element 2314A, a conductor which is electrically connected to a respective one of the more distally-located stimulation elements, such as stimulation element 2314B, terminates at that location. This arrangement is repeated throughout the length of the stimulation portion 2311 (in a distal orientation) such that by the time the most distal stimulation element (e.g. 2314F) is reached, just one electrical signal conductor 2317 extends within and through the conduit. Accordingly, as the stimulation portion extends distally from the most proximal stimulation element 2314A, each respective lead segment 2315B, 2315C (and so on) may, in some examples, become progressively more flexible than the preceding lead segment (e.g. 2315A, 2315B, and so on) because fewer conductors 2317 are present within the conduit. This progressively increasing flexibility distally along the length of the stimulation portion 2311 may enhance the ability to introduce, advance, and/or maneuver the respective more distal stimulation elements and corresponding lead segments in and among target tissues. Whether alone or in combination with other described features and attributes of the example of FIGS. 29A-29B, this progressively increasing flexibility may promote independent positioning of the stimulation elements 2314A, 2314B, etc. relative to each other and/or relative to the different target nerve portions, muscle portions, etc.

Via such arrangements, in some examples, a flexibility of the stimulation portion 2311 may increase from about 30 to 50 percent from the most proximal stimulation element 2314A to the most distal stimulation element 2314F.

In contrast, at least some other non-example lead designs may maintain substantially uniform relative flexibility (e.g. stiffness) distally toward a distal tip of a lead in order to enable pushability of such leads through a tunnel or vasculature to place a stimulation electrode (or sensing electrode) at a desired location within patient anatomy. Such stiffness would significantly inhibit the type, manner, and extent of maneuverability contemplated for the example stimulation portion of the examples of the present disclosure, as further described in association with.

With this in mind, one example method of implantation may comprise forming a tunnel, via the implant-access incision 609A, to advance and position more proximal portions of the lead body for connection to a pulse generator (e.g. 217 in FIG. 1B) while more distal portions of the lead, such as the distal lead portion 2313 including the stimulation portion 2311 comprising the connected array of stimulation elements 2314A-2314F may be placed in and among target tissues without little or no tunneling to achieve an implanted configuration as shown in FIG. 29A, for example. In this arrangement, portions of the lead 2310 which are proximal to distal lead portion 2313 may comprise a stiffness to enable advancement, pushability, etc. within and through a tunnel. A surgical accessory may be used to form the tunnel and deliver the lead, similar to an introducer. Conversely, because in some examples the distal lead portion 2313 is not being advanced through a tunnel (in some examples), the distally increasing flexibility of the stimulation portion 2311 may enhance maneuverability, positioning, etc. of the stimulation elements 2314A, 2314B, etc. independently of each other relative to the target tissues.

With further reference to at least FIG. 29A, at least one of the respective lead segments 2315A-2315F may comprise a shape, size, and/or length, which enables the particular lead segment 2315A-2315F to exhibit a variable length to provide strain relief for a particular stimulation element, the stimulation portion 2311 as a whole, the distal lead portion 2313, and/or the lead 2310 as a whole. With this in mind, as shown in FIG. 29A, in one non-limiting example, lead segments 2315C, 2315D, and 2315F comprise a length between adjacent pairs of stimulation elements such that a loop is formed in each respective lead segment 2315C, 2315D, 2315F to provide the above-noted strain relief. It will be understood that just one, just some, or all of the lead segments 2315A-2315F may comprise such a loop to provide a variable length property (e.g. loop) or other shape to provide a variable length, such as later shown in FIG. 29C.

In some examples, the lead segment 2315D may comprise an overall length which is substantially greater than a length of the other respective lead segments (e.g. 2315B, 2315C, 2315E, 2315F) in order to provide sufficient length to straddle the sagittal midline 316 to enable positioning the respective stimulation elements in stimulating relation to target tissues on opposite sides (e.g. 312R, 312L) of the patient's body, while also enabling maneuverability of the stimulation elements 2314A, 2314B, etc. independently of each other in relation to specific target tissues. In some examples, the length of lead segment 2315D is substantially greater than the length of the other respective lead segments (e.g. 2315B, 2315C, etc.). In some such examples, this substantially greater difference may comprise a difference of at least 25 percent, at least 50 percent, at least 75 percent, at least 100 percent, at least 150 percent, or at least 200 percent.

FIG. 29C is a top plan view schematically representing an example portion 2307 which may comprise one example implementation of at least one of the respective lead segments (e.g. 2315A, 2315B, etc.). As shown in FIG. 29C, the example portion 2307 may comprise one or more undulating curved segments 2308 and/or other shapes which enable the example portion 2307 to have a variable length, which may provide strain relief for a particular lead segment (e.g. between an adjacent pair of stimulation elements), the stimulation portion 2311 as a whole, and/or at least the distal lead portion 2313 of the lead 2310. The undulating shape may comprise a shape (e.g. sinusoidal, sigmoid, other) which repeats or non-repeating arcuate segments. In some examples, the variable length portion 2307 of FIG. 29C may comprise one alternative example implementation of the example lead segments 2315C, 2315D, and/or 2315F in FIG. 29A.

FIGS. 29D-29I provide a series of illustrations of various example stimulation elements. In some examples, the various stimulation elements described in FIGS. 29D-29I may comprise at least some of substantially the same features and attributes as, may be example implementations of, and/or may be consistent with the stimulation elements (and related arrangements) described in association with various example stimulation elements described throughout the present disclosure. In one example, the stimulation elements 2314A-2314F of FIG. 29A may be implemented via one or more of the example arrangements of stimulation elements in FIGS. 29D-29I or may be implemented via other arrangements of a carrier and contact electrodes.

FIG. 29D is a top plan view schematically representing example stimulation element 3200. As shown in FIG. 29D, example stimulation element 3500 comprises a paddle electrode 3510 comprising a paddle-style body 3512 on which a linear array 3514 of electrodes 3516 are located. In some examples, the array 3514 may comprise a two-dimension array of electrodes 3516. The paddle electrode 3510 is supported by, and extends from, a distal portion 3520 of a stimulation lead 3522.

As further shown in the side view of FIG. 29E, the paddle electrode 3510 may be positioned in stimulating relation to a nerve 3528, such as a hypoglossal nerve (e.g. 505R) or IHM-innervating nerve (e.g. 515R) or other nerve related to increasing and/or maintaining upper airway patency. The paddle electrode 3510 may be secured in pressing contact with the nerve 3528 or may be secured in close proximity to, but spaced apart from, the nerve 3528.

FIG. 29F is a side plan view schematically representing an example arrangement 3541 in which stimulation element 3540 is in stimulating relation to a nerve 3528 (like in FIG. 29E). In some examples, the stimulation element 3540 may comprise at least some of substantially the same features and attributes as stimulation element 3510, except with electrodes 3516 being arranged in a linear array 3545 of spaced apart ring electrodes 3546. In some examples, the carrier supporting the electrodes 3516 may comprise a substantially cylindrical shape.

FIG. 29G is a side plan view schematically representing an example arrangement 3561 in which stimulation element 3560 is adapted to be in stimulating relation to a nerve (like nerve 3528 in FIGS. 29E, 29F). In some examples, the stimulation element 3560 may comprise at least some of substantially the same features and attributes as stimulation element 3540 in FIG. 29F, except comprising a linear array 3565 of spaced apart circumferentially extending electrodes 3566 instead of ring electrodes 3546 in FIG. 29F and with body 3542 comprising a generally cylindrical shape. In some such examples, the spaced apart circumferentially extending electrodes 3566 may comprise split-ring electrodes.

FIG. 29H is a side view, and FIG. 291 is a side view, schematically representing an example arrangement 3600 including a cuff electrode 3630. In some examples, cuff electrode 3630 comprises a bottom row of axially spaced apart electrodes 3636D and a middle row of circumferentially spaced apart electrodes 3636A, 3636B, 3636D, 3636C. By independently activating various combinations of the respective electrodes 3636A, 3636B, 3636C, 3636D, as well as variations in the stimulation signal as previously described, this electrode configuration may be used to provide selective stimulation and/or stimulation steering of a stimulation signal relative to different fascicles, nerve fibers, etc. within a nerve about which the cuff electrode 3630 is secured. In some such examples, the cuff electrode 3630 comprises one or more rows of electrodes (e.g. 3636D) aligned parallel to a longitudinal axis of the carrier of the cuff electrode 3630, without including the circumferentially arrangement of electrodes (e.g. 3636A, 3636B, 3636D, and/or 3636C). Conversely, in some such examples, the cuff electrode 3630 comprises one or more rows of electrodes (e.g. 3636A, 3636B, 3636C, and/or 3636D) aligned perpendicular (or at some other angle) relative to a longitudinal axis of the carrier of the cuff electrode 3600 without including the longitudinally extending arrangement of electrodes (e.g. multiple 3636D). In some examples, the circumferentially arranged electrodes may have different lengths from each other, may be offset from each other in a longitudinal orientation along the length of the cuff, and/or may embody other spacing variations.

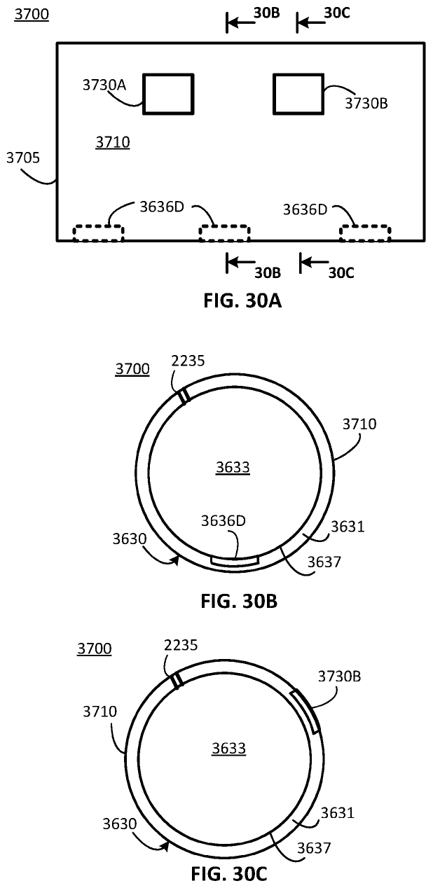

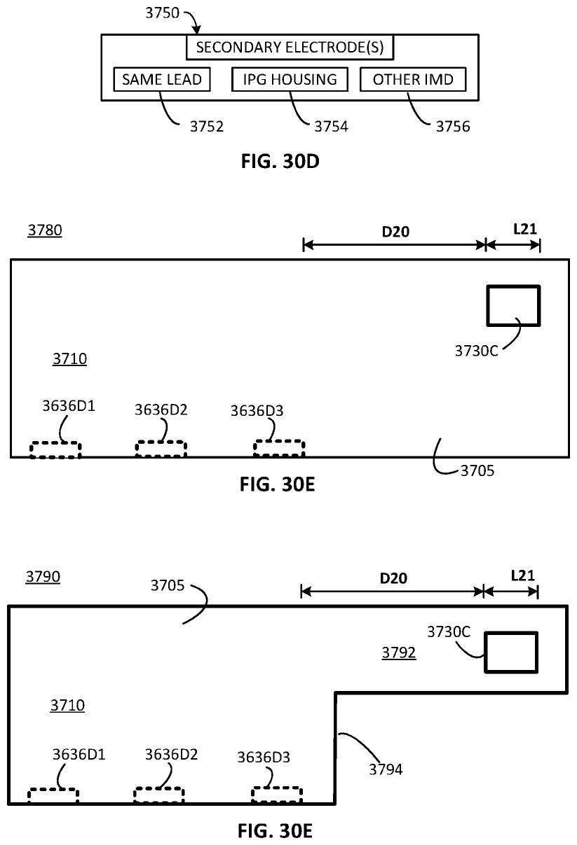

FIGS. 30A-30C illustrate an example electrode arrangement 3700 and related aspects (FIGS. 30D-30F). In some examples, the electrode arrangement 3700 may comprise an example implementation of, and/or comprise at least some of substantially the same features as, various example electrode arrangements in association with at least FIGS. 1A-291, 31-36, except with an additional electrode(s) (e.g. 3730A and/or 3730B) exposed on an external surface of an electrically non-conductive carrier of an electrode arrangement. The additional electrode(s) may sometimes be referred to as a secondary electrode. In some examples, the additional electrode may be used for sensing, stimulation, and/or other purposes.

With this context in mind, FIG. 30A is a front view of an example cuff-style electrode arrangement akin to the electrode arrangement 3600/3630 of FIG. 29H-291, except comprising at least one additional electrode (e.g. 3730A and/or 3730B) present on an external surface portion 3710 of a cuff body 3705 (e.g. generally cylindrical member to at least partially encircle a nerve). As seen in FIG. 30A, cuff body 3705 supports electrodes 3636D (shown in dashed lines) on an internal surface exposed within a lumen (e.g. 3633 in FIGS. 30B, 30C) of the cuff body 3705, which may be used for sensing and/or stimulation of a first nerve N1 in a manner similar to that described in association with at least FIGS. 1A-29I. However, being present on external surface portion 3710 of cuff body 3705, the additional electrodes 3730A, 3730B are necessarily isolated from direct engagement with first nerve N1 while being exposed to surrounding tissues external to the cuff body 3705. In some examples, the surrounding tissues may comprise muscles and/or nerves related to a function also served by the first nerve N1 being engaged via the internal electrodes 3636D of the cuff electrode arrangement 3700. For example, the first nerve N1 may comprise an upper airway patency-related nerve for maintaining or promoting upper airway patency while at least some of the surrounding tissues also may be related to respiration and/or particularly related to maintaining or promoting upper airway patency. In some such examples, the nerve engaged via the internal electrodes 3636D may comprise the hypoglossal nerve. In some such examples, the surrounding tissues may be various tissues within a neck region from which respiration sensing may be performed via sensing impedance using additional electrodes 3730A and/or 3730B, which in some examples, also may be used in conjunction with other electrodes implanted within the neck region for sensing impedance. These other electrodes may sometimes be referred to as secondary electrodes 3750, as shown in FIG. 30D, with such secondary electrode(s) 3750 being located on a different portion of the same lead 3752 which supports cuff electrode arrangement 3700, located on an IPG housing 3754 in communication with the cuff electrode arrangement 3700, and/or located on another implantable medical device (IMD) separate from, and independent of, the IMD including the cuff electrode arrangement 3700. With such secondary electrode(s) 3750 present within the neck region, the additional electrode(s) 3730A, 3730B may be used to sense bio-impedance from tissues between the additional electrode(s) 3730A, 3730B and the secondary electrodes 3750, and the sensed bio-impedance used to sense respiration.

In some examples, via the additional electrodes 3730A, 3730B alone or in conjunction with secondary electrode(s) 3750, other parameters may be sensed such as electromyography (EMG) signals from muscles immediately adjacent or between such electrodes. Such EMG information may provide respiratory information, such as respiratory phase information, disease burden information, upper airway patency information, and/or other physiologic information.

In some examples, instead of being implanted relative to the hypoglossal nerve, the cuff-style electrode arrangement 3700 may be implanted to at least partially encircle other upper airway patency-related nerves such as, but not limited to, an infrahyoid muscle (IHM)-innervating nerve to be electrically engaged by electrodes 3636D while external additional electrode(s) 3730A, 3730B may be in operative relation to (e.g. sensing and/or stimulating relation) other tissue such as (but not limited to) a respiratory-related tissue. In some such examples, the other tissue may comprise a phrenic nerve from which respiration may be sensed via at least electrodes 3730A, 3730B with or without secondary electrode(s) 3750 (FIG. 30D). The respiratory information sensed via the phrenic nerve may be used to perform closed loop stimulation therapy in which stimulation periods are synchronized relative to sensed respiratory phase information, sensed respiratory morphology, etc. such as the stimulation periods coinciding with the inspiratory phase, in some examples.

In some examples, regardless of whether such closed loop stimulation therapy or open loop stimulation therapy (e.g. stimulation not synchronized relative to sensed respiratory information) is performed, the sensed respiratory information may be used to evaluate therapy efficacy such as via monitoring whether disease burden (e.g. apnea burden, such as via an apnea-hypopnea index (AHI)) increases or decreases with particular stimulation therapy protocols, parameters, settings (e.g. amplitude), etc.

Accordingly, in such examples, the cuff-style electrode arrangement 3700 including internal electrodes 3636D and external electrode(s) 3730A, 3730B (FIG. 30D) may be used to be in operative relation with two different tissues (e.g. respiratory tissues), which are in reasonably close proximity while performing two different tasks, i.e. the internal electrodes 3636D for stimulating a first type of nerve (e.g. IHM-innervating nerve) and the external electrodes 3730A, 3730B for sensing a second type of nerve (e.g. phrenic nerve).

In some examples, the cuff-style electrode arrangement 3700 may be used in a converse manner in which the cuff body 3705 at least partially encircles a first nerve for electrodes 3636D to be operative relation (e.g. sensing and/or stimulating relation) with the first nerve and the external electrodes 3730A, 3730B to be in operative relation (e.g. sensing and/or stimulating relation) with a second tissue. In some examples, the first nerve may comprise a phrenic nerve from which respiration may be sensed via electrodes 3636D and/or by which activation of the diaphragm muscle may be performed. In some examples, stimulation of the second tissue via external electrode(s) 3730A, 3730B may maintain or promote upper airway patency. In some such examples, the second tissue may comprise an infrahyoid muscle (IHM) and/or IHM-innervating nerve.

However, in some examples in which the first nerve comprises a phrenic nerve and the internal electrodes 3636D are used to sense respiration, the external electrodes 3730A, 3730B also may engage a second tissue to obtain additional or further respiratory information to augment the respiratory information obtained via sensing the phrenic nerve. In some examples, the external electrodes 3730A, 3730B may be used to sense solely the second tissue engaged by such electrodes or, the external electrodes 3730A, 3730B may engage the second tissue but perform sensing in association with other tissues in the neck region to sense impedance, EMG, etc. from which respiration and/or other physiologic information may be determined.

With regard to the examples of FIGS. 30A-30D, which also may be representative of similar embodiments for the wraps of FIGS. 7-19 and/or other cuff-style electrode arrangements, the placement of additional electrodes (e.g. 3730A, 3730B) on an external surface portion 3710 of the cuff body 3705 acts to electrically isolate the sensing activity from the stimulation signals being applied via the internal electrodes (e.g. 3636D in FIG. 30A), which may lessen interference between the lower magnitude (e.g. finer) sensing signals (e.g. bioimpedance, etc.) and the higher magnitude (e.g. large) stimulation signals.

With this context in mind, as shown in FIG. 30E, in some examples, cuff body 3705 may be sized and/or shaped to locate the additional electrode(s) 3730C on external surface portion 3710 to be spaced apart by a distance D20 from the nearest stimulation electrode (e.g. 3636D3) located on the internal surface of the cuff body. The distance D20 may comprise a distance suitable to minimize such interference. In some examples, the distance D20 may comprise a selectable quantity (e.g. 10 millimeters), a multiple of a length (L21) of electrode 3730C (or length of electrode 3636D3), and/or a distance set by other criteria. In some such examples, the multiple may comprise at least 3, 4, or 5 times the length L21 of electrode 3730C or 3636D3.

In the illustrated example of FIG. 30F, instead of the entire cuff body 3705 being extended to achieve the target distancing (e.g. D20) of the external electrode 3730C from the nearest internal electrode (e.g. 3636D3), just a portion of the cuff body 3705 forms an extension 3792 extending (e.g. longitudinally in some examples) from a first end portion 3794 of cuff body 3705, which otherwise would normally define the entire end of cuff body 3705 in the absence of extension 3792. It will be understood that the location of the extension 3792 is not limited to the example shown in FIG. 30E, and that in some examples the extension 3792 may instead extend from any portion of cuff body 3705 such as (but not limited to) a lower portion of cuff body 3705 aligned with internal electrodes 3636D1, 3636D3, 3636D3.

Regardless of whether a configuration like FIG. 30E or FIG. 30F is employed, the position of the electrode 3730C shown in the example of FIG. 30E as being on an opposite/upper side of the cuff body 3705 increases the effective distance from the nearest internal electrode (e.g. 3636D3) and may further minimize the interference from any stimulation signals emitted from the nearest internal electrode (e.g. 3636D3).

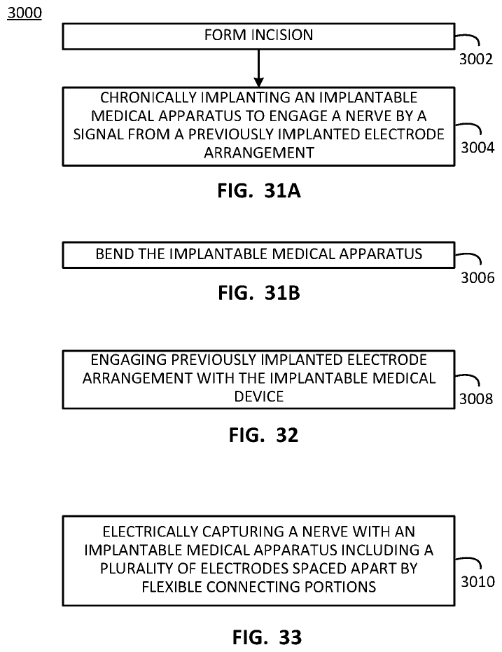

FIGS. 31A-32 illustrate various example methods 3000 of the disclosure and via which at least some of the various examples of FIGS. 1A-30C may be implemented. Some methods include accessing the previously implanted first electrode arrangement includes creating an incision at 3002 according to examples of the disclosure. As shown at 3004 in FIG. 31A, various examples include a method of chronically implanting an implantable medical apparatus (e.g., any of FIGS. 1A, 3A-27) to achieve electrical capture of target tissue by a signal (stimulation or sensing) from a previously implanted first electrode arrangement.

The target tissue may comprise nerve portions, muscle portions, combinations of nerve portions and muscle portions, neuromuscular junctions of nerve portions and muscle portions, and/or combinations thereof. In some examples, the target tissues may comprise a nerve portion innervating the intrinsic muscles of the tongue, the intrinsic muscle portions of the tongue, and/or neuromuscular junctions of the nerve portion and intrinsic tongue muscles portion. With this in mind, the target tissue may increase or maintain upper airway patency to treat sleep disordered breathing (SDB) such as (but not limited to) obstructive sleep apnea. Among other examples, stimulation of the hypoglossal nerve may cause contraction of at least tongue protrusor muscles, thereby resulting in tongue protrusion to at least partially increase or maintain upper airway patency. Meanwhile, stimulation of at least some infrahyoid-muscle (IHM) innervating nerves may cause movement of other upper airway structures to increase or maintain upper airway patency. In some examples, the target tissue may comprise other nerves and/or muscles contributing to upper airway patency.

In some examples, as shown at 3006 in FIG. 31B, the implantable medical apparatus may be bent/manipulated at a target site to achieve the desired positioning. In examples where the implantable medical apparatus includes at least one cuff, methods may include positioning each cuff around at least one nerve. Example methods include anchoring the implantable medical apparatus in position within a patient, which can include securing an anchor to tissue, which may or may not be adjacent to the nerve.

As further shown at 3008 in FIG. 32, example methods may include engaging the previously implanted electrode arrangement with the implantable medical apparatus. In various examples, the step of engaging includes inserting the previously implanted first electrode arrangement at least partially into a dock of the implantable medical apparatus. In some example methods, the previously implanted first electrode arrangement is connected to an electrode arrangement of the dock. In one example, the step of pulling the previously implanted first electrode arrangement into the dock is accomplished at least in part with an elongated member. In some examples in which the implantable medical apparatus including a second electrode arrangement and a third electrode arrangement, the method may comprise engaging the previously implanted first electrode arrangement with the third electrode arrangement and/or securing the second electrode arrangement of the implantable medical apparatus to the target nerve. Various methods may include securing a wrap of the implantable medical apparatus around the previously implanted first electrode arrangement. One example includes securing at least one tether of the implantable medical apparatus to the previously implanted electrode arrangement. Some example methods include a step of positioning the previously implanted electrode arrangement within a groove of the wrap. In some example methods, the method comprises securing the wrap or a plurality of wraps around a circumference of the target nerve. In some example methods, the method comprises securing the wrap or a plurality of wraps around a previously implanted electrode arrangement. One example method comprises securing a wrap over electrodes of the previously implanted electrode arrangement and securing at least one clip to the previously implanted first electrode arrangement to interconnect the previously implanted electrode arrangement and the wrap. In one example, the plurality of wraps are secured to the previously implanted first electrode arrangement such that the plurality of wraps collectively cover all of the electrodes of the previously implanted first electrode arrangement.

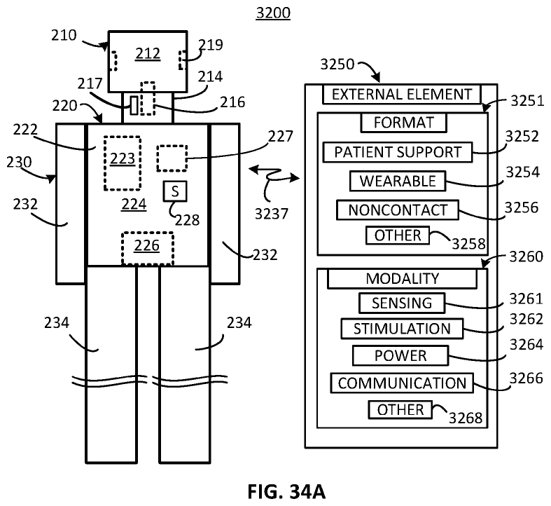

FIG. 33 is a block diagram illustrating an example method of the disclosure. One example method includes chronically implanting an implantable medical apparatus (for example, but not limited to, any of FIGS. 1A, 24, 26, 29A), to electrically capture target tissue at 3010. In various examples, the implantable medical apparatus includes a plurality of electrodes spaced apart by flexible connecting portions, which may be made of a nonconductive material. In some examples, the implantable medical apparatus may receive a signal (e.g. stimulation) from a previously implanted first electrode arrangement. In various examples, the method includes wrapping a portion of the implantable medical apparatus around the target tissue, the target tissue being a nerve. In some examples, the portion is wrapped around a nerve more than 360 degrees. In some examples, at least part of the portion is wrapped in a helical fashion around the target nerve (e.g., see FIGS. 24-25). In various examples, the implantable medical apparatus is connected to a plurality of different nerve branches (see also, FIG. 25 and related disclosure).

An example arrangement 3200 of the disclosure, in which one or more example implantable medical apparatuses of the disclosure can be incorporated, is shown in FIG. 34A. Various examples of the present disclosure have described various aspects of chronically implantable electrode arrangements which may be used for sensing and/or for stimulation and which may be implemented in association with a pulse generator, which is shown as being implanted, such as pulse generator 533 in FIG. 2A or pulse generator 2180 in FIG. 27. However, it will be understood that any one of the various previously described, example implantable medical apparatuses (e.g. electrode arrangements and associated components (e.g. docks, additional electrode arrangements, etc.)) may be implemented, as represented via sensing element(s) 228 and/or stimulation element(s) 217, to be in wireless communication (e.g. connection 3237) with at least one external element 3250.

As further shown in FIG. 34A, in some examples, the external element(s) 3250 may be implemented via a wide variety of formats such as, but not limited to, at least one of the formats 3251 including a patient support 3252 (e.g. bed, chair, sleep mat, other), wearable elements 3254 (e.g. finger, wrist, head, neck, shirt), noncontact elements 3256 (e.g. watch, camera, mobile device, other), and/or other elements 3258.

As further shown in FIG. 34A, in some examples, the external element(s) 3250 may comprise one or more different modalities 3260 such as (but not limited to) a sensing portion 3261, stimulation portion 3262, power portion 3264, communication portion 3266, and/or other portion 3268. The different portions 3261, 3262, 3264, 3266, 3268 may be combined into a single physical structure (e.g. package, arrangement, assembly), may be implemented in multiple different physical structures, and/or with just some of the different portions 3261, 3262, 3264, 3266, 2268 combined together in a single physical structure. In some examples, at least some aspects of the sensing portion 3261, stimulation portion 3262, power portion 3264, and/or communication portion 3266 may function as pulse generation components and be located externally provide at least some aspects of stimulation signals and/or sensing signals instead of such stimulation and/or sensing being provided via pulse generators 533 (FIGS. 2A), 2180 (FIG. 27) which were implanted in the previously described examples. Such externally provided sensing and/or stimulation functions may be communicated (e.g. to and/or from) relative to the various implanted sensing element(s) 228 and/or stimulation element(s) 217 via wireless communication. It will be further understood that in some examples, some components supporting and implementing such sensing and/or stimulation (via the implanted electrode arrangements) may be located externally, while some components supporting and implementing such sensing and/or stimulation (via the implanted electrode arrangements) may be implanted.

In some examples, the external power portion 3264 and/or power components associated with implanted stimulation element 217 may comprise at least some of substantially the same features and attributes of at least the stimulation arrangements, as described throughout the present disclosure. In some such examples, the respective power portion, components, etc. may comprise a rechargeable power element (e.g. source, battery, circuitry elements) and/or non-rechargeable power elements (e.g. battery). In some examples, the external power portion 3264 may comprise a power source by which a power component of the implanted stimulation element 217 may be recharged.

In some examples, the wireless communication portion 3266 (e.g. including connection/link at 3237) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3268 via which other aspects of implementing medical care may be embodied in external element(s) 3250 to relate to the various implanted and/or external components described above.

As previously noted in various examples of the disclosure, in some examples an upper airway patency-related nerve may comprise an IHM-innervating nerve in addition to, or instead of, a hypoglossal nerve.

In some examples, an IHM-innervating nerve may comprise a nerve or nerve branch which innervates (directly or indirectly) at least one infrahyoid muscle, which may sometimes be referred to as an infrahyoid strap muscle. In some examples, IHM-innervating nerves/nerve branches extend from (e.g. originates) from a nerve loop called the ansa cervicalis (AC) or the “AC loop nerve”, which stems from the cervical plexus, e.g. extending from cranial nerves C1-C3. Accordingly, in some examples, at least some IHM-innervating nerves may correspond to an ansa cervicalis (AC)-related nerve in the sense that such nerves/nerve branches (e.g. IHM-innervating nerves) do not form the AC loop nerve but extend from the AC loop nerve. At least because the AC loop nerve is the origin for some nerves which innervate muscles other than the infrahyoid muscles, some AC-related nerves do not comprise IHM-innervating nerves. Moreover, it will be understood that in some examples, stimulation applied to a portion (e.g., superior root) of the AC loop nerve (and/or to nerves from which the AC loop nerve originates) may activate IHM-innervating nerves/nerve branches, which extend from the AC loop nerve. However, implementing stimulation (e.g. to influence upper airway patency) occurring at more proximal locations, such as along the superior root of the AC loop nerve may be more complex because of the number/type of different nerves and number/type of different muscles innervated via a superior root of the AC loop nerve such that selective activation of a particular infrahyoid muscle (via stimulation along the superior root) may be quite challenging in some circumstances.

With this background in mind, FIG. 34B is a diagram 9900 schematically representing patient anatomy and providing further details regarding example devices and/or example methods for stimulating an IHM-innervating nerve and/or hypoglossal nerve. As shown in FIG. 34B, diagram 9900 includes a side view schematically representing an AC-main nerve 9915, in context with a hypoglossal nerve 9905 and with cranial nerves C1, C2, C3. As shown in FIG. 34B, portion 9929A of the AC-main nerve 9915 (e.g. a portion or trunk connecting to the AC loop nerve 9919) extends anteriorly from a first cranial nerve C1 with a segment 9917 running alongside (e.g. coextensive with) the hypoglossal nerve 9905 for a length until the AC-main nerve 9915 diverges from the hypoglossal nerve 9905 to form a superior root 9925 of the AC-main nerve 9915, which forms part of the AC loop nerve 9919. A portion of the hypoglossal nerve 9905 extends distally to innervate the genioglossus muscle 9904. As further shown in FIG. 34B, the superior root 9925 of the AC-main nerve 9915 extends inferiorly (i.e. downward) until reaching near bottom portion 9918 of the AC loop nerve 9919, from which the AC loop nerve 9919 extends superiorly (i.e. upward) to form an lesser root 9927 (i.e. inferior root) which joins to the second and third cranial nerves, C2 and C3, respectively and via portions 9929B, 9929C.

As further shown in FIG. 34B, several branches 9931 extend off the AC loop nerve 9919, including branch 9932 which innervates the omohyoid muscle group 9934, branch 9942 which innervates the sternothyroid muscle group 9944 and at least a portion (e.g. inferior portion) of the sternohyoid muscle group 9954. Another branch 9952, near bottom portion 9918 of the AC loop nerve 9919, innervates at least a portion (e.g. superior portion) of the sternohyoid muscle group 9954. In some examples, the collective arrangement of the AC-main nerve 9915 (including at least superior root 9925 of the AC loop nerve 9919) and its related branches (e.g. at least 9932, 9942, 9952) when considered together, or any of those elements individually, may sometimes be referred to as an IHM-innervating nerve 9916. It will be further understood that at least one such IHM-innervating nerve 9916 is present on both sides (e.g. right and left) of the patient's body.

In some examples, stimulation of the superior root 9925 of AC loop nerve 9919 and/or at least some of the branches 9931 extending from the AC loop nerve 9919, may influence upper airway patency. However, in some examples, upper airway patency also may be increased and/or maintained by directly stimulating the above-identified muscle groups, such as the omohyoid, sternothyroid, and/or sternohyoid muscle groups. Accordingly, in some examples, such stimulation also may comprise stimulation of just a nerve portion(s), just muscle portion(s), a combination of nerve portion(s) and muscle portion(s), a neuromuscular junction of nerve portion(s) and muscle portion(s), and combinations thereof. Among other effects, in some examples stimulation of such nerves and/or muscles (and/or neuromuscular junctions, combinations, etc.) may act to bring the larynx inferiorly, which may increase upper airway patency.

Stimulation may be delivered to many different locations of an IHM-innervating nerve 9916/nerve branches. Of these various potential stimulation locations, FIG. 34B generally illustrates three example stimulation locations A, B, and C. A stimulation element may be placed at all three of these locations or just some (e.g. one or two) of these example stimulation locations. At each location, a wide variety of types of stimulation elements (e.g. cuff electrode, axial array, paddle electrode, etc.) may be implanted depending on the particular delivery path, method, etc. For example, any one or a combination of the various example stimulation elements (and associated manner of access, delivery, etc.) described in association with at least FIGS. 1A-25 may be used to deliver such stimulation. In some such examples, a scale of the various stimulation elements, anchors, access tools, and/or other elements in some of the examples in FIGS. 1A-25L may be reduced to accommodate a generally smaller diameter of the IHM-innervating nerve/nerve branches 9916 as compared to some other nerve portions, such as at least some portions of the hypoglossal nerve.

With further reference to FIG. 34B, at each example stimulation A, B, C, a stimulation element may be delivered subcutaneously, intravascularly, etc. At each stimulation location, in some examples the stimulation element may comprise a microstimulator.

FIG. 35A is a block diagram schematically representing an example control portion 4000. In some examples, control portion 4000 provides one example implementation of a control portion forming a part of, implementing, and/or generally managing stimulation elements (e.g. including electrode arrangements), power/control elements (e.g. pulse generators, microstimulators), sensors, and related elements, devices, user interfaces, instructions, information, engines, elements, functions, actions, and/or methods, as described throughout examples of the present disclosure in association with FIGS. 1-34 and 35B-36.

In some examples, control portion 4000 includes a controller 4002 and a memory 4010. In general terms, controller 4002 of control portion 4000 comprises at least one processor 4004 and associated memories. The controller 4002 is electrically couplable to, and in communication with, memory 4010 to generate control signals to direct operation of at least some of the stimulation elements, power/control elements (e.g. pulse generators, microstimulators), sensors, and related elements, devices, user interfaces, instructions, information, engines, elements, functions, actions, and/or methods, as described throughout examples of the present disclosure. In some examples, these generated control signals include, but are not limited to, employing instructions 4011 and/or information 4012 stored in memory 4010 to at least perform sensing, stimulation, and/or other actions, functions, etc. as part of providing sleep disordered breathing care or other therapies, as described throughout the examples of the present disclosure in association with FIGS. 1-34 and 35B-36. In some instances, the controller 4002 or control portion 4000 may sometimes be referred to as being programmed to perform the above-identified actions, functions, etc. such that the controller 4002, control portion 4000 and any associated processors may sometimes be referred to as being a special purpose computer, control portion, controller, or processor. In some examples, at least some of the stored instructions 4011 are implemented as, or may be referred to as, a care engine, a sensing engine, monitoring engine, and/or treatment engine. In some examples, at least some of the stored instructions 4011 and/or information 4012 may form at least part of, and/or, may be referred to as a care engine, sensing engine, monitoring engine, and/or treatment engine (e.g. stimulation engine).

In response to or based upon commands received via a user interface (e.g. user interface 4040 in FIG. 35C) and/or via machine readable instructions, controller 4002 generates control signals as described above in accordance with at least some of the examples of the present disclosure. In some examples, controller 4002 is embodied in a general purpose computing device while in some examples, controller 4002 is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of this application, in reference to the controller 4002, the term “processor” shall mean a presently developed or future developed processor (or processing resources) that executes machine readable instructions contained in a memory. In some examples, execution of the machine readable instructions, such as those provided via memory 4010 of control portion 4000 cause the processor to perform the above-identified actions, such as operating controller 4002 to implement the sensing, monitoring, determining, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 4010. In some examples, the machine readable instructions may comprise a sequence of instructions, a processor-executable machine learning model, or the like. In some examples, memory 4010 comprises a computer readable tangible medium providing non-volatile storage of the machine readable instructions executable by a process of controller 4002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 4002 may be embodied as part of at least one application-specific integrated circuit (ASIC), at least one field-programmable gate array (FPGA), and/or the like. In at least some examples, the controller 4002 is not limited to any specific combination of hardware circuitry and machine readable instructions, nor limited to any particular source for the machine readable instructions executed by the controller 4002.

In some examples, control portion 4000 may be entirely implemented within or by a stand-alone device.

In some examples, the control portion 4000 may be partially implemented in one of the sensing devices, monitoring devices, stimulation devices, apnea treatment devices (or portions thereof), etc. and partially implemented in a computing resource separate from, and independent of, the apnea treatment devices (or portions thereof) but in communication with the apnea treatment devices (or portions thereof). For instance, in some examples control portion 4000 may be implemented via a server accessible via the cloud and/or other network pathways. In some examples, the control portion 4000 may be distributed or apportioned among multiple devices or resources such as among a server, an apnea treatment device (or portion thereof), and/or a user interface.

In some examples, control portion 4000 includes, and/or is in communication with, a user interface 4040 as shown in FIG. 35C.

FIG. 35B is a diagram schematically illustrating at least some example implementations of a control portion 4020 by which the control portion 4000 (FIG. 35A) can be implemented, according to one example of the present disclosure. In some examples, control portion 4020 is entirely implemented within or by a pulse generator (PG) assembly 4025, which has at least some of substantially the same features and attributes as a pulse generator (e.g. power/control element, microstimulator) as previously described throughout the present disclosure. In some examples, control portion 4020 is entirely implemented within or by a remote control 4030 (e.g. a programmer) external to the patient's body, such as a patient control 4032 and/or a physician control 4034. In some examples, the control portion 4000 is partially implemented in the IPG assembly 4025 and partially implemented in the remote control 4030 (at least one of patient control 4032 and physician control 4034).

FIG. 35C is a block diagram schematically representing user interface 4040, according to one example of the present disclosure. In some examples, user interface 4040 forms part or and/or is accessible via a device external to the patient and by which the therapy system may be at least partially controlled and/or monitored. The external device which hosts user interface 4040 may be a patient remote (e.g. 4032 in FIG. 35B), a physician remote (e.g. 4034 in FIG. 35B) and/or a clinician portal. In some examples, user interface 4040 comprises a user interface or other display that provides for the simultaneous display, activation, and/or operation of at least some of the stimulation elements, power/control elements (e.g. pulse generators, microstimulators), sensors, and related elements, devices, user interfaces, instructions, information, engines, functions, actions, and/or method, etc., as described in association with FIGS. 1-35B and 36. In some examples, at least some portions or aspects of the user interface 4040 are provided via a graphical user interface (GUI), and may comprise a display 4044 and input 4042.

FIG. 36 is a block diagram 4300 which schematically represents some example implementations by which a device (MD) 4310 (e.g. implantable in some example), such as a pulse generator and/or sensing monitor, may communicate wirelessly with other devices. In some examples, the other devices may be located outside the patient. As shown in FIG. 36, in some examples, the MD 4310 may communicate with at least one of patient app 4330 on a mobile device 4320, a patient remote control 4340, a clinician programmer 4350, and a patient management tool 4336. The patient management tool 4336 may be implemented via a cloud-based portal 4362, the patient app 4330, and/or the patient remote control 4340. Among other types of data, these communication arrangements enable the MD 4310 to communicate, display, manage, etc. sleep/wake data for patient management as well as to allow for adjustment to the detection method if/where needed.

It will be understood that at least some of the various devices/elements 4320, 4340, 4350, patient management tool 4360 also may communicate with each other, with or without communicating with the implantable device 4310.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-64. (canceled)

65. An implantable medical apparatus comprising:
a lead body;
a first electrode arrangement connected to the lead body and comprising a plurality of electrodes in an axial-style electrode arrangement;
a second electrode arrangement configured to be controlled via the operation and control of the plurality of electrodes of the first electrode arrangement; and
a third electrode arrangement configured to engage with the first electrode arrangement to electrically couple the first electrode arrangement to the second electrode arrangement.

66. The implantable medical apparatus of claim 65, wherein the third electrode arrangement comprises a dock configured to receive the first electrode arrangement.

67. The implantable medical apparatus of claim 66, wherein the dock includes at least one seal.

68. The implantable medical apparatus of claim 66, wherein the dock includes a first opening at a first end and a second opening at a second end.

69. The implantable medical apparatus of claim 68, wherein the first opening and the second opening are aligned with a central longitudinal axis of the dock.

70. The implantable medical apparatus of claim 65, wherein the second electrode arrangement comprises a cuff electrode.

71. The implantable medical apparatus of claim 65, wherein the second electrode arrangement comprises a paddle electrode.

72. An implantable medical apparatus comprising:
a lead body;
a first electrode arrangement connected to the lead body and comprising a plurality of electrodes in an axial-style electrode arrangement;
a second electrode arrangement configured to be controlled via the operation and control of the plurality of electrodes of the first electrode arrangement;
a third electrode arrangement configured to engage with the first electrode arrangement to electrically couple the first electrode arrangement to the second electrode arrangement; and
a lead body extension positioned between the second electrode arrangement and the third electrode arrangement.

73. The implantable medical apparatus of claim 72, wherein the lead body extension includes an anchor.

74. The implantable medical apparatus of claim 72, wherein the lead body extension is flexible.

75. The implantable medical apparatus of claim 72, wherein the lead body extension includes a bendable section including a pre-formed non-linear shape.

76. The implantable medical apparatus of claim 72, wherein the lead body extension extends from a side of, and at an angle relative to a longitudinal axis of the third electrode arrangement.

77. An implantable medical apparatus comprising:
an electrode arrangement comprising a lead connected to a plurality of electrodes in an axial-style electrode arrangement; and
a first wrap configured to be attached to the electrode arrangement and positioned around a circumference of the electrode arrangement.

78. The implantable medical apparatus of claim 77, wherein the first wrap spans at least 360 degrees.

79. The implantable medical apparatus of claim 77, wherein the first wrap comprises an interior surface comprising a groove to receive the electrode arrangement.

80. The implantable medical apparatus of claim 77, further comprising:
a second wrap spaced apart from the first wrap and a first tether extending between, and connecting, the first wrap to the second wrap; and
a third wrap spaced apart from the first wrap opposite to the second wrap and a second tether extending between, and connecting, the first wrap to the third wrap.

81. The implantable medical apparatus of claim 80, wherein the first tether and the second tether comprise a rigid material.

82. The implantable medical apparatus of claim 80, wherein the first tether and the second tether comprise a flexible material.

83. The implantable medical apparatus of claim 77, wherein the electrode arrangement comprises at least one retainer.

84. The implantable medical apparatus of claim 83, further comprising:
at least one clip to secure the first wrap to the at least one retainer.
